(19) United States
(12) Patent Application Publication
(10) Pub. No.: US 2026/0340774 A1
(43) Pub. Date: Oct. 29, 2026

(54) METHOD AND SYSTEM FOR ACCELERATING MAGNETIC RESONANCE IMAGING

(71) Applicants: Ramot at Tel-Aviv University Ltd., Tel-Aviv (IL); Tel HaShomer Medical Research Infrastructure and Services Ltd., Ramat-Gan (IL)

(72) Inventors: Gali HOD, Tel-Aviv (IL); Nahum KIRYATI, Tel-Aviv (IL); Arnaldo MAYER, Ramat HaSharon (IL); Ilya NELKENBAUM, Tirat HaCarmel (IL); Alexander NAZAROV, Tel-Aviv (IL)

(73) Assignees: Ramot at Tel-Aviv University Ltd., Tel-Aviv (IL); Tel HaShomer Medical Research Infrastructure and Services Ltd., Ramat-Gan (IL)

(21) Appl. No.: 19/119,787

(22) PCT Filed: Oct. 11, 2023
(86) PCT No.: PCT/IL2023/051069

§ 371 Date: Apr. 10, 2025

Related U.S. Application Data
(60) Provisional application No. 63/415,015, filed on Oct. 11, 2022.

(51) Int. Cl. G06T 5/50; G01R 33/56

(52) U.S. Cl. CPC G06T 5/50; G01R 33/5601; G06T 2207/10088; G06T 2207/20081; G06T 2207/20084

(57) ABSTRACT

A method of magnetic resonance imaging (MRI) comprises: acquiring a first MRI scan having a first type of MR image contrast, and a second MRI scan having a second type of MR image contrast. The acquisition ensures that the two MRI scans are blurred along different phase encoding directions. The method also comprises feeding a machine learning procedure trained to deblur MR images with input data describing the first and the second MRI scans, and receiving from an output of the machine learning procedure output data corresponding to a deblurred MR image.

### RELATED APPLICATION

This application claims the benefit of priority of U.S. Provisional Patent Application No. 63/415,015 filed on Oct. 11, 2022, the contents of which are incorporated herein by reference in their entirety.

### FIELD AND BACKGROUND OF THE INVENTION

The present invention, in some embodiments thereof, relates to magnetic resonance imaging and, more particularly, but not exclusively, to a method and a system for accelerating magnetic resonance imaging.

Magnetic Resonance Imaging (MRI) is a method to obtain an image representing the chemical and physical microscopic properties of materials, by utilizing a quantum mechanical phenomenon, named Nuclear Magnetic Resonance (NMR), in which a system of spins, placed in a magnetic field resonantly absorb energy, when applied with a certain frequency.

A nucleus can experience NMR only if its nuclear spin I does not vanish, i.e., the nucleus has at least one unpaired nucleon. Examples of non-zero spin nuclei frequently used in MRI include ¹H (I=1/2), ²H (I=1), ²³Na (I=3/2), etc. When placed in a magnetic field, a nucleus having a spin I is allowed to be in a discrete set of energy levels, the number of which is determined by I, and the separation of which is determined by the gyromagnetic ratio of the nucleus and by the magnetic field. Under the influence of a small perturbation, manifested as a radiofrequency magnetic field, which rotates about the direction of a primary static magnetic field, the nucleus has a time dependent probability to experience a transition from one energy level to another. With a specific frequency of the rotating magnetic field, the transition probability may reach the value of unity. Hence at certain times, a transition is forced on the nucleus, even though the rotating magnetic field may be of small magnitude relative to the primary magnetic field. For an ensemble of spin I nuclei the transitions are realized through a change in the overall magnetization.

Once a change in the magnetization occurs, a system of spins tends to restore its magnetization longitudinal equilibrium value, by the thermodynamic principle of minimal energy. The time constant which control the elapsed time for the system to return to the equilibrium value is called “spin-lattice relaxation time” or “longitudinal relaxation time” and is denoted T₁. An additional time constant, T₂ (≤T₁), called “spin-spin relaxation time” or “transverse relaxation time”, controls the elapsed time in which the transverse magnetization diminishes, by the principle of maximal entropy. However, inter-molecule interactions and local variations in the value of the static magnetic field, alter the value of T₂, to an actual value denoted T₂*.

In MRI, a static magnetic field having a predetermined gradient is applied on an object, thereby creating, at each region of the object, a unique magnetic field. There are typically three types of gradient fields that are used in MRI: a slice-selective gradient, a frequency-encoding gradient, and a phase-encoding gradient. The slice-selective gradient selects a specific slice or cross-section of the object. This gradient is applied along a direction in space, altering the strength of the magnetic field along that direction, leading to a variation in the resonance frequency of the nuclei within the slice. The frequency-encoding gradient is typically applied perpendicularly to the slice-selective gradient in order to distinguish the resonant frequencies of nuclei within the selected slice, allowing to determining the position of the signal along an axis in the image (known as the frequency-encoding axis, or the readout axis). The phase-encoding gradient provides additional spatial encoding along an axis that is known as the phase-encoding axis and that is perpendicular to both the slice-selective and frequency-encoding gradients. The phase-encoding gradient allows the MRI system to differentiate nuclei in the same frequency range along the phase-encoding axis.

In MRI, pulse sequences are applied to the object (e.g., a patient) to generate NMR signals and obtain information therefrom which is subsequently used to reconstruct images of the object. The above mentioned relaxation times and the density distribution of the nuclear spin are properties which vary from one normal tissue to the next, and from one diseased tissue to the next. These quantities are therefore responsible for contrast between tissues in various imaging techniques, hence permitting image segmentation.

Over the past decade, compressed sensing (CS) techniques have been successfully applied to MRI data acquisition, leading to significant scanning time reductions [Lustig, et al., Magnetic Resonance in Medicine 58(6), 1182-1195 (2007); https://doi.org/https://doi.org/10.1002/mrm.2139120, Lustig, et al., IEEE Signal Processing Magazine 25(2), 72-82 (2008)]. CS is a mathematical framework developed for data reconstruction from highly under-sampled data by leveraging sparse representations. In CS-MRI, the image is recovered from semi-random incomplete sampling of k-space data using an appropriate nonlinear recovery scheme.

### SUMMARY OF THE INVENTION

According to some embodiments of the invention the present invention there is provided a method of magnetic resonance imaging (MRI). The method comprises: acquiring a first MRI scan having a first type of MR image contrast according to a first anisotropic acquisition matrix having less elements along a first direction than along a second direction, the first direction being a phase-encoding direction for the first MRI scan. The method also comprises acquiring a second MRI scan having a second type of MR image contrast according to a second anisotropic acquisition matrix having less elements along the second direction than along the first direction, the second direction being a phase-encoding direction for the second MRI scan. The method also comprises feeding a machine learning procedure trained to deblur MR images with input data describing the first and the second MRI scans, and receiving from an output of the machine learning procedure output data corresponding to a deblurred MR image.

According to some embodiments of the invention the method comprises, prior to the feeding, co-registering the first and the second MRI scans.

According to some embodiments of the invention the first and the second MRI scans are acquired, respectively, before and after administration of a contrast agent to the object.

According to some embodiments of the invention the first type of MR image contrast is different from the second type of MR image contrast, and wherein each of the first and the second type of MR image contrasts is selected from the group consisting of a T1-Weighted MR image contrast, a T2-Weighted MR image contrast, a Proton Density Weighted MR image contrast, a Fluid-Attenuated Inversion Recovery MR image contrast, a Diffusion-Weighted MR image contrast, and an Inversion Recovery MR image contrast.

According to some embodiments of the invention a number of elements of the first anisotropic acquisition matrix along the second direction equals a number of elements of the second anisotropic acquisition matrix along the first direction.

According to some embodiments of the invention a number of elements of the first anisotropic acquisition matrix along the first direction equals a number of elements of the second anisotropic acquisition matrix along the second direction.

According to some embodiments of the invention each of the first and the second anisotropic acquisition matrix are three-dimensional and comprise elements also along a third direction, the third direction being a phase-encoding direction.

According to some embodiments of the invention a number of elements in the second three-dimensional anisotropic acquisition matrix along the third direction is less than a number of elements in the first three-dimensional anisotropic acquisition matrix along the third direction, and wherein a number of elements in the first three-dimension anisotropic acquisition matrix along the second direction is less than a number of elements in the second three-dimension anisotropic acquisition matrix along the first second.

According to some embodiments of the invention each of the first and the second anisotropic acquisition matrices are three-dimensional and comprise elements also along a third direction, wherein the method comprises acquiring a third MRI scan having a third type of MR image contrast according to a third anisotropic three-dimensional acquisition matrix having fewest elements along the third direction among the first, the second and the third three dimensional acquisition matrices, and wherein the input data also describe the third MRI scan.

According to some embodiments of the invention the method comprises receiving from the machine learning procedure output data corresponding to two or more deblurred MR images, at least one deblurred MR image for each of the MR scan.

According to some embodiments of the invention the machine learning procedure comprises an artificial neural network, and wherein each of at least two of the branches has an anisotropic kernel which is enlarged along direction corresponding to a direction of a respective acquisition matrix along which the matrix has less elements.

According to an aspect of some embodiments of the present invention there is provided a method of MRI. The method comprises: acquiring a first MRI scan having a first type of MR image contrast according to a first radial acquisition protocol, and acquiring a second MRI scan having a second type of MR image contrast according to a second radial acquisition protocol, wherein radial sampling lines describing the first radial acquisition protocol are interlaced with radial sampling lines describing the second radial acquisition protocol. The method also comprises feeding a machine learning procedure trained to deblur MR images with input data describing the first and the second MRI scans, and receiving from an output of the machine learning procedure output data corresponding to a deblurred MR image.

According to some embodiments of the invention the first and the second MRI scans are acquired, respectively, before and after administration of a contrast agent to the object.

According to some embodiments of the invention the first type of MR image contrast is different from the second type of MR image contrast, and wherein each of the first and the second type of MR image contrasts is selected from the group consisting of a T1-Weighted MR image contrast, a T2-Weighted MR image contrast, a Proton Density Weighted MR image contrast, a Fluid-Attenuated Inversion Recovery MR image contrast, a Diffusion-Weighted MR image contrast, and an Inversion Recovery MR image contrast.

According to some embodiments of the invention the method comprises receiving from the machine learning procedure output data corresponding to two or more deblurred MR images, at least one deblurred MR image for each of the MR scan.

According to some embodiments of the invention the method comprises acquiring at least one additional MRI scan having at least one respective type of MR image contrast according to a respective at least one additional radial acquisition protocol, wherein radial sampling lines describing each of the first, the second, and the at least one additional radial acquisition protocols, are all interlaced cyclically thereamongst, wherein the input data also describe the at least one additional MRI scan.

According to an aspect of some embodiments of the present invention there is provided a method of MRI. The method comprises: acquiring a first MRI scan having a first type of MR image contrast according to a first spiral acquisition protocol, and acquiring a second MRI scan having a second type of MR image contrast according to a second spiral acquisition protocol, wherein spirals describing the first spiral acquisition protocol are interlaced with spirals describing the second spiral acquisition protocol. The method also comprises feeding a machine learning procedure trained to deblur MR images with input data describing the first and the second MRI scans, and receiving from an output of the machine learning procedure output data corresponding to a deblurred MR image.

According to some embodiments of the invention the first and the second MRI scans are acquired, respectively, before and after administration of a contrast agent to the object.

According to some embodiments of the invention the first type of MR image contrast is different from the second type of MR image contrast, and wherein each of the first and the second type of MR image contrasts is selected from the group consisting of a T1-Weighted MR image contrast, a T2-Weighted MR image contrast, a Proton Density Weighted MR image contrast, a Fluid-Attenuated Inversion Recovery MR image contrast, a Diffusion-Weighted MR image contrast, and an Inversion Recovery MR image contrast.

According to some embodiments of the invention method comprises receiving from the machine learning procedure output data corresponding to two or more deblurred MR images, at least one deblurred MR image for each of the MR scan.

According to some embodiments of the invention the machine learning procedure comprises a plurality of branches, each receiving all the input data but providing output data corresponding to one of the two or more deblurred MR images.

According to some embodiments of the invention the method comprises acquiring at least one additional MRI scan having at least one respective additional type of MR image contrast according to at least one respective additional spiral acquisition protocol, wherein spirals describing each of the first, the second, and the at least one additional spiral acquisition protocols, are all interlaced cyclically thereamongst, wherein the input data also describe the at least one additional MRI scan.

According to an aspect of some embodiments of the present invention there is provided a computer software product. The computer software product comprises a computer-readable medium in which program instructions are stored, which instructions, when read by an image processor, cause the data processor to receive input data, to feed a machine learning procedure trained to deblur MR images with the input data, and to receive from an output of the machine learning procedure output data corresponding to a deblurred MR image. According to some embodiments of the invention the input data comprise data describing a first MRI scan having a first type of MR image contrast according to a first anisotropic acquisition matrix having less elements along a first direction than along a second direction, the first direction being a phase-encoding direction for the first MRI scan. According to some embodiments of the invention the input data also comprise a second MRI scan having a second type of MR image contrast according to a second anisotropic acquisition matrix having less elements along the second direction than along the first direction, the second direction being a phase-encoding direction for the second MRI scan.

According to some embodiments of the invention a number of elements of the first anisotropic acquisition matrix along the second direction equals a number of elements of the second anisotropic acquisition matrix along the first direction.

According to some embodiments of the invention a number of elements of the first anisotropic acquisition matrix along the first direction equals a number of elements of the second anisotropic acquisition matrix along the second direction.

According to some embodiments of the invention each of the first and the second anisotropic acquisition matrix are three-dimensional and comprise elements also along a third direction, the third direction being a phase-encoding direction.

According to some embodiments of the invention a number of elements in the first three-dimensional anisotropic acquisition matrix along the third direction is less than a number of elements in the second three-dimensional anisotropic acquisition matrix along the third direction, and wherein a number of elements in the second three-dimension anisotropic acquisition matrix along the second direction is less than a number of elements in the first three-dimension anisotropic acquisition matrix along the first direction.

According to some embodiments of the invention a number of elements in the second three-dimensional anisotropic acquisition matrix along the third direction is less than a number of elements in the first three-dimensional anisotropic acquisition matrix along the third direction, and wherein a number of elements in the first three-dimension anisotropic acquisition matrix along the second direction is less than a number of elements in the second three-dimension anisotropic acquisition matrix along the second direction.

According to some embodiments of the invention each of the first and the second anisotropic acquisition matrices are three-dimensional and comprise elements also along a third direction, wherein the method comprises acquiring a third MRI scan having a third type of MR image contrast according to a third anisotropic three-dimensional acquisition matrix having fewest elements along the third direction among the first, the second and the third three dimensional acquisition matrices, and wherein the input data also describe the third MRI scan.

According to an aspect of some embodiments of the present invention there is provided a computer software product. The computer software product comprises a computer-readable medium in which program instructions are stored, which instructions, when read by an image processor, cause the data processor to receive input data, to feed a machine learning procedure trained to deblur MR images with the input data, and to receive from an output of the machine learning procedure output data corresponding to a deblurred MR image. According to some embodiments of the invention the input data comprises data describing a first MRI scan having a first type of MR image contrast according to a first radial acquisition protocol, and a second MRI scan having a second type of MR image contrast according to a second radial acquisition protocol, wherein radial sampling lines describing the first radial acquisition protocol are interlaced with radial sampling lines describing the second radial acquisition protocol.

According to some embodiments of the invention the interlacing forms a radial pattern having a uniform angular spacing between adjacent radial sampling lines of the pattern.

According to an aspect of some embodiments of the present invention there is provided a computer software product. The computer software product comprises a computer-readable medium in which program instructions are stored, which instructions, when read by an image processor, cause the data processor to receive input data, to feed a machine learning procedure trained to deblur MR images with the input data, and to receive from an output of the machine learning procedure output data corresponding to a deblurred MR image. According to some embodiments of the invention the input data comprises data describing a first MRI scan having a first type of MR image contrast according to a first spiral acquisition protocol, and a second MRI scan having a second type of MR image contrast according to a second spiral acquisition protocol, wherein spirals describing the first spiral acquisition protocol are interlaced with spirals describing the second spiral acquisition protocol.

According to some embodiments of the invention the interlacing forms a spiral pattern having a uniform spacing between adjacent spirals of the pattern.

According to an aspect of some embodiments of the present invention there is provided a magnetic resonance imaging (MRI) system for imaging an object. The system comprises an MRI scanner configured for scanning the object to provide MRI scans, and an image processor configured for executing the method as delineated above and optionally and preferably as further detailed below.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C are flowchart diagrams describing methods suitable for magnetic resonance imaging (MRI), according to some embodiments of the present invention;

FIGS. 2A-D are schematic illustrations showing examples of a grid suitable for use according to some embodiments of the present invention;

FIG. 3 is a schematic illustration of an MR scanner system for imaging an object, according to some embodiments of the present invention;

FIGS. 4A-C are schematic illustrations of a registration process, which can be employed according to some embodiments of the present invention;

FIG. 5 is a schematic illustration of a machine learning procedure, according to some embodiments of the present invention;

FIG. 6 is a schematic illustration of a branched machine learning procedure, according to some embodiments of the present invention;

FIG. 7 is a collection of MR images as obtained in experiments performed according to some embodiments of the present invention;

FIG. 8 is another collection of MR images as obtained in experiments performed according to some embodiments of the present invention;

FIGS. 9A and 9B show quantitative performance comparison in terms of peak signal-to-noise ratio (FIG. 9A) and Structural Similarity index (FIG. 9B), for T1 weighted scans following administration of a contrast agent, as obtained in experiments performed according to some embodiments of the present invention;

FIGS. 10A and 10B show quantitative performance comparison in terms of peak signal-to-noise ratio (FIG. 9A) and Structural Similarity index (FIG. 9B), for T1 weighted scans without contrast agent, as obtained in experiments performed according to some embodiments of the present invention;

FIG. 11 is a schematic illustration of three different phase-encoding directions which can be used in three-dimensional MRI, according to some embodiments of the present invention;

FIG. 12 is a schematic illustration of a machine learning procedure which can be used when MRI acceleration is executed along more than two axes, according to some embodiments of the present invention;

FIG. 13 is a schematic illustration of a branched machine learning procedure which can be used when MRI acceleration is executed along more than two axes, according to some embodiments of the present invention;

FIGS. 14A and 14B are schematic illustrations describing MRI acceleration for a radial grid, according to some embodiments of the present invention; and

FIG. 14C is a schematic illustration describing MRI acceleration for a spiral grid, according to some embodiments of the present invention.

### DESCRIPTION OF SPECIFIC EMBODIMENTS OF THE INVENTION

The present invention, in some embodiments thereof, relates to magnetic resonance imaging and, more particularly, but not exclusively, to a method and a system for accelerating magnetic resonance imaging.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

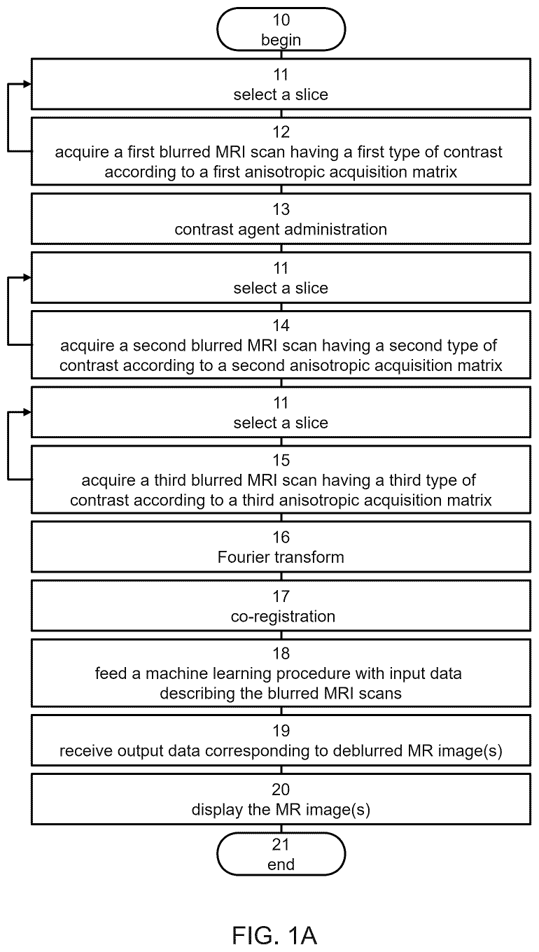

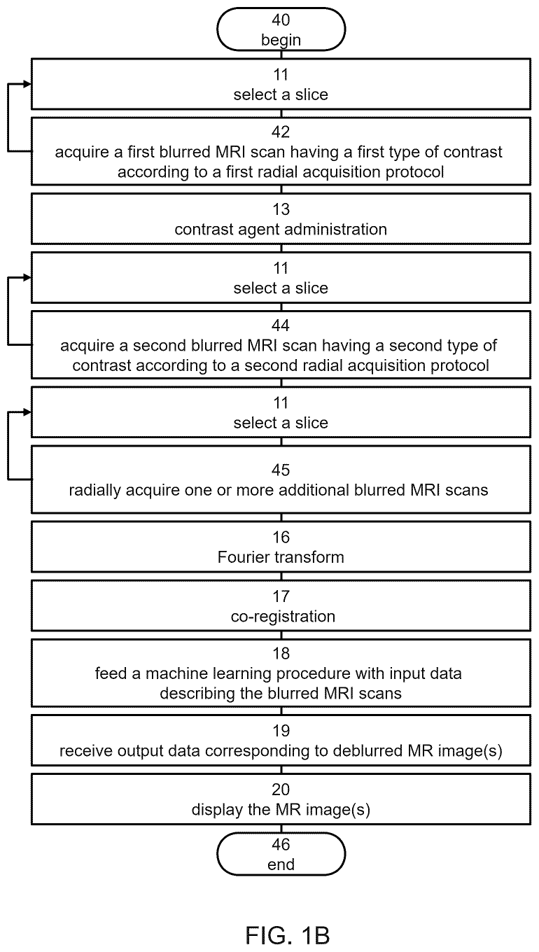

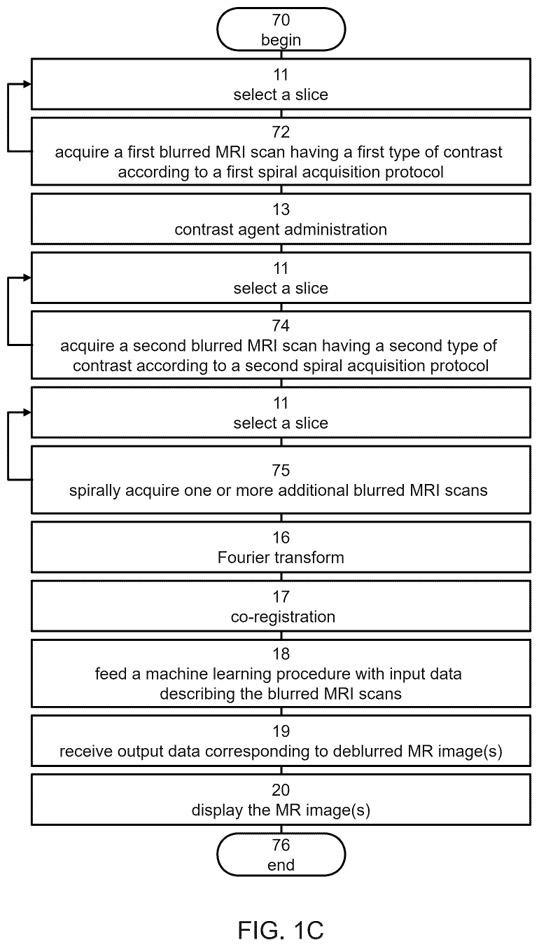

Referring now to the drawings, FIGS. 1A-C are flowchart diagrams describing methods suitable for magnetic resonance imaging (MRI), according to some embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations described herein can be implemented by a data processing system, e.g., a dedicated circuitry or a general purpose processor, configured for executing the operations described below. At least part of the operations can be implemented by a cloud-computing facility at a remote location.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pull these data structures or values for use in subsequent operation. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

Selected operations of the method optionally and preferably can be executed by a magnetic resonance (MR) scanner having a controller configured for activating a radiofrequency transmitter system for generating a pulse sequence, and a gradient assembly for generating gradient fields, including a slice-selective gradient, a frequency-encoding gradient, and a phase-encoding gradient. The MR scanner also comprises an acquisition system for acquiring MR signals responsively to the pulse sequence generated by the radiofrequency transmitter system.

The method of the present embodiments is particularly useful for MRI of objects such as an organ or a full body of a living human or animal, but embodiments in which the method is used for MRI of a non-living object are also contemplated.

Referring to FIG. 1A, the method begins at 10 and optionally and preferably continues to 11 at which a slice of the object is selected. This can be done by the controller of the MR system that can select the slice by generating a gradient field that ensures that only nuclei in the selected slice are in phase and contribute to the MR signal, while nuclei outside the slice are out of phase and contribute less or not at all. It is to be understood that operation 11 is optional and that in some embodiments of the present invention it is not executed. For example, when it is desired to construct a two-dimensional MR image there is no need to apply slice selection.

The method continues to 12 at which a first MRI scan having a first type of MR image contrast is acquired by the MR scanner. Representative examples of types of MR image contrast suitable for use according to some embodiments of the present invention include, without limitation, a T1-Weighted (T1W) MR image contrast, a T2-Weighted (T2W) MR image contrast, a Proton Density (PD) Weighted MR image contrast, a Fluid-Attenuated Inversion Recovery (FLAIR) MR image contrast, a Diffusion-Weighted MR image contrast, and an Inversion Recovery (IR) MR image contrast. Each of these types of image contrast can be achieved by a judicious construction of the pulse sequence to be generated by the radiofrequency transmitter system of the MR scanner. Thus, the first MRI scan is acquired by transmitting to the object a first pulse-sequence and receiving from the object an MR signal in response to this pulse-sequence.

The acquisition of the first MRI scan is according to a first acquisition matrix. The acquisition matrix can be a two-dimensional matrix or a three-dimensional matrix. The matrix comprises a plurality of matrix-elements arranged along a first direction and a second direction. In embodiments in which the acquisition matrix is three-dimensional its matrix-elements are arranged also along a third direction. When the matrix is two-dimensional one of the directions of the matrix is a phase-encoding direction and the other direction is a readout or frequency-encoding direction. When the matrix is three-dimensional, there are typically two phase-encoding directions and one readout direction. Thus, the MR scanner scans the object according to the acquisition matrix such that when one moves along a phase-encoding direction each element of the matrix corresponds to a different phase shift in the MR signal allowing to differentiate nuclei within the same frequency bin along this direction, and when one moves along a frequency-encoding direction each element of the matrix corresponds to a different bin of resonance frequencies for the same phase shift of the MR signal.

Without loss of generality, the first direction of the acquisition matrix is referred to below as “direction x,” the second direction of the acquisition matrix is referred to below as “direction y,” and the third direction of the acquisition matrix is referred to below as “direction z.”

The acquisition matrix is preferably defined over a space known as “the k-space” of the MRI. The k-space represents spatial frequencies rather than physical locations. In a k-space, coordinate values that are smaller in their absolute value correspond to lower spatial frequencies (and therefore to lower resolution) than coordinate values that are higher in their absolute value. The acquisition matrix represents a discretization of a Cartesian k-space over a grid that includes a plurality of discrete grid cells (each corresponding to one matrix-element of the acquisition matrix), where the positions of the grid cells are determined by the strength and timing of the applied gradients. Once data is collected in the k-space, an MR image can be reconstructed by a mathematical transform called a Fourier transform, which converts the data from the spatial frequency domain of the k-space into the spatial domain of the image space.

While the acquisition matrix defined above represents a discretization of the k-space over a Cartesian system of coordinates, use of Cartesian system of coordinates is not necessary, since, as will be explained hereinunder, it may be desired to acquire MR scans also over a non-cartesian k-space grid.

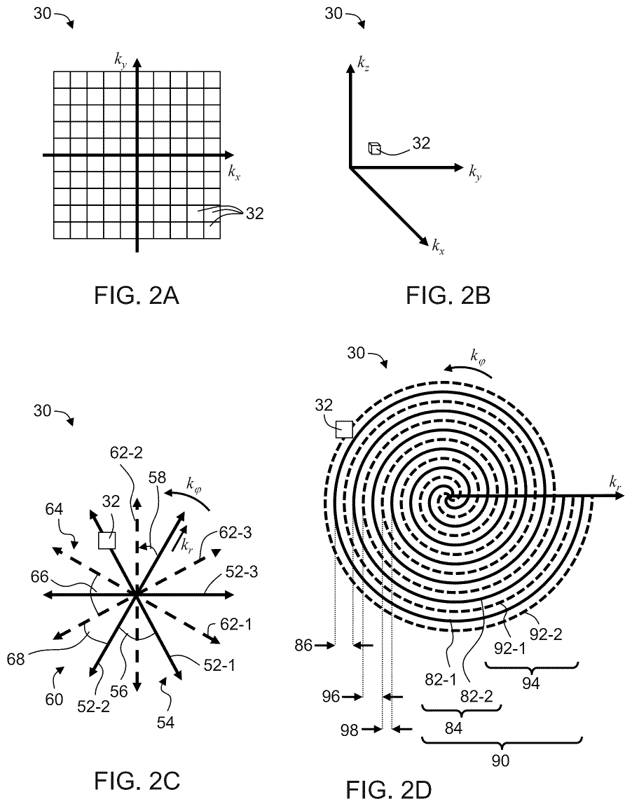

FIGS. 2A-D illustrates examples of a grid 30 suitable for use according to some embodiments of the present invention. The cells 32 of grid 30 represent the discretization of the space over which the grid is defined. For clarity of presentation a plurality of cells 32 of grid 30 are only shown in FIG. 2A, whereas in each of FIGS. 2B-D only one representative cell 32 is illustrated. FIGS. 2A and 2B illustrate examples in which the grid is a Cartesian grid, in which case the grid can be defined over two (FIG. 2A) or three (FIG. 2B) orthogonal axes, respectively corresponding to a two-dimensional grid and three-dimensional grid. Thus, the cells 32 in FIG. 2A represent the matrix-elements of an acquisition matrix when the matrix is a two-dimensional matrix, and the cells 32 (only one is illustrated) in FIG. 2B represent the matrix-elements of an acquisition matrix when the matrix is a three-dimensional matrix.

FIGS. 2C and 2D illustrate examples in which the grid 30 is a non-Cartesian grid, in which case the grid 30 can be defined over a plurality of directions that are not necessarily orthogonal to each other and are not necessarily straight. FIG. 2C illustrates an example of a two-dimensional radial grid and FIG. 2D illustrates an example of a two-dimensional spiral grid. The skilled person, provided with the details described herein would appreciate that three-dimensional versions of the grid types shown in FIGS. 2C and 2D can also be employed.

It is appreciated that the radial grid in FIG. 2C has a plurality of non-parallel radial directions kᵣ, wherein an azimuthal coordinate k_{φ} is defined along circular lines perpendicular to the radial axes, so that for a given distance from the origin, along any of the radial directions, the azimuthal coordinate varies from 0 to 2π. In case of the spiral grid (FIG. 2D) there is no need to define radial directions kᵣ (although these can be defined as illustrated in FIG. 2D) because each cell can be defined by means of a single coordinate value φ.

Other types of non-Cartesian grids, such as, but not limited to, a barycentric grid, elliptic grid, hyperbolic grid, etc., are also contemplated in some embodiments of the present invention.

A particular feature of the present embodiments is that the first acquisition matrix is anisotropic. The matrix is “anisotropic” in the sense that the number of elements are not the same in all the directions. For example, in the first acquisition matrix, there are less elements along direction x than along the direction y. In this embodiment, the first MRI scan is acquired while employing phase-encoding along direction x. When the first acquisition matrix is a two-dimensional matrix, the first MRI scan is acquired while employing frequency encoding along direction y. It is appreciated that in the two-dimensional case, the density of phase-encoding is lower than the density of frequency encoding because there are less matrix-elements along direction x. The case in which the first acquisition matrix is a three-dimensional matrix is explained in greater detail hereinunder.

It is also appreciated that since the numbers of matrix-elements differ along different directions, the acquisition time of the first MRI scan is accelerated compared to the acquisition time that would have been required had the numbers of matrix-elements been the highest along all directions. On the other hand, due to the reduced number of elements along one or more of the directions there is a loss of information along these directions, resulting in a blurring of the corresponding MR image along the respective direction(s) in the image space. The MRI scan is therefore referred to in FIG. 1A as a “blurred” to indicate the aforementioned information loss. Yet, as will be explained hereinunder, and as demonstrated in the Examples section that follows, the inventors found that this loss of information can be compensated by acquiring one or more additional blurred MRI scans.

Referring again to FIG. 1A, from 12 the method optionally and preferably loops back to 11 for selecting another slice of the object, and repeating the acquisition 12 for the newly selected slice. This loop can continue until the acquisition is completed for all the slices of the object. When operation 11 is not executed, there is no need to loop back from 12 and the method continues as further detailed hereinbelow.

In some embodiments of the present invention the method continues to 13 at which a detectable dose of an MR contrast agent is administered to the subject. Alternatively, the method can begin while the subject already has the contrast agent in his or her vasculature, in which case operation 13 is not executed. Still alternatively, the method can be executed in its entirety without the use of contrast agent, in which case operation 13 is also not executed. When operation 13 is executed the administered contrast agent can be of any type that enhances the contrast among different materials in the imaged object, e.g., by changing one or more of the relaxation times T₁, T₂, T₂*. The MR contrast agent can be either a positive or a negative MRI contract agent.

As used herein, “positive MR contract agent” refers to an agent which increases the signal relative to nearby materials, and “negative MR contract agent” refers to an agent which decreases the signal relative to nearby materials.

The magnetic properties of the MR contrast agent can be of any type. More specifically, the MRI contrast agent comprises a magnetic material which can be paramagnetic, superparamagnetic or ferromagnetic material. Representative examples of MR contrast agents suitable for the present embodiments include, without limitation, gadolinium (Gd), dysprosium (Dy), chromium (Cr), iron (Fe), and manganese (Mn), more preferably Gd, Mn, and Fe, and most preferably Gd.

Operation 13, when executed, can be executed before or after operations 11 and 12.

Following the execution of 12 and optionally following the execution of 13, the method optionally and preferably re-execute 11 to select a slice. This operation is only executed in cases in which operation 11 is executed before 12. Otherwise, this operation can be skipped.

At 14, a second MRI scan is acquired by the MR scanner according to a second acquisition matrix, which is different from the first acquisition matrix. The second MRI scan is arranged over a grid of coordinates of the same type as the grid of coordinate over which the first MRI scan is arranged. For example, when the first MRI scan is acquired by scanning a two-dimensional Cartesian k-space grid, the second MRI scan is also acquired by scanning a two-dimensional Cartesian k-space grid, and when the first MRI scan is acquired by scanning a three-dimensional Cartesian k-space grid, the second MRI scan is also acquired by scanning a three-dimensional Cartesian k-space grid.

The second MRI scan has a second type of MR image contrast. The second type of MR image contrast can be any of the aforementioned types of MR image contrast. As stated, the type of image contrast can be achieved by a judicious construction of the pulse sequence to be generated by the radiofrequency transmitter system of the MR scanner, and so the second MRI scan is typically acquired by transmitting to the object a second pulse-sequence and receiving from the object an MR signal in response to this pulse-sequence.

At least one of the acquisition conditions is preferably different among operations 12 and 14. This difference can be in terms of the contrast agent that may or may not be present in the object, and/or in terms of the type of MR image contrast that is selected by means of the pulse sequence applied before the acquisition. These two types of differences are referred to collectively as difference in the modality of the MRI. When operation 13 is employed, and when operation 12 is executed before operation 13, the contrast agent is not present in the object during operation 12 but is present in the object during operation 14, and so the first and second MR image contrasts can, but not necessarily, be of the same type. For example, both the first and the second MRI scans can be T1W MRI scans, or T2W MRI scans and so no, except that the first MRI scan is acquired without the presence of contrast agent in the object, and the second MRI scan is acquired with the presence of contrast agent in the object.

When operation 13 is not employed, or is executed before operation 12, there is no difference among the acquisition conditions in terms of the contrast agent, and so the first and second MR image contrasts are of different types. For example, the first MRI scan can be a T1W MRI scan, and the second MRI scan can be a T2W MRI scan, and so on.

Similarly to operation 12 above, the second MRI scan is also blurred in the sense that it was acquired by a second acquisition matrix which is anisotropic. Yet, unlike the first acquisition matrix, the second acquisition matrix has less elements along direction y than along direction x, and the second MRI scan is acquired while employing phase-encoding along direction y. When the second acquisition matrix is a two-dimensional matrix the second MRI scan is acquired while employing frequency encoding along direction x. Thus, similarly to the first MRI scan, the density of phase-encoding is lower than the density of frequency encoding, except that for the second MRI scan the phase-encoding and frequency encoding directions are swapped relative to these directions in the first MRI scan.

In case in which the first and second acquisition matrices are three-dimensional matrices, any possible combination of two different directions among x, y, z (namely, any of the pairs: x-y, x-z, y-z, z-x, z-y, y-x) can be selected such that the first direction of the pair has less elements in the first matrix than in the second matrix whereas the second direction of said pair has less elements in the second matrix than in the first matrix, where the first and second directions are phase encoding direction in the first and second acquisition matrices, respectively

From 14 the method optionally and preferably loops back to 11 for selecting another slice of the object, and repeating the acquisition 14 for the newly selected slice. This loop can continue until the acquisition is completed for all the slices of the object. This operation is only executed in cases in which operation 11 is executed before 12. Otherwise, this operation can be skipped.

Following is a description of embodiments of the invention in which the first and second acquisition matrices are three-dimensional matrices, defined over the directions x, y, and z. In this case, the matrices can be used for the acquisition of 3D MRI. This can be done in more than one way.

In some embodiments of the present invention the z direction of each of the first and second acquisition matrices is a phase-encoding direction, so that for each of the first and second MRI scans there are two phase-encoding directions (directions x and z in the first MRI scan, and directions y and z in the second MRI scan). The remaining direction, as the case may be (direction y in the first MRI scan, and direction x in the second MRI scan) is a frequency encoding direction.

In some embodiments of the present invention the number of matrix-elements along direction z is less for the first acquisition matrix than for the second acquisition matrix. This corresponds to a case in which the extent of blurring along direction z is higher for the first MRI scan than for the second MRI scan. Preferably, in these embodiments the number of elements in the second matrix along direction y is less than the number of elements in the first matrix along direction y. Alternatively, the number of elements in the second matrix along direction x is less than the number of elements in the first matrix along direction x.

Alternatively, the number of matrix-elements along direction z can be less for the second acquisition matrix than for the first acquisition matrix, corresponding to a case in which the extent of blurring along direction z is higher for the second MRI scan than for the first MRI scan. Preferably, in these embodiments the number of elements in the first matrix along direction y is less than a number of elements in the second matrix along direction y. Also contemplated are embodiments in which the number of elements in the first matrix along direction x is less than the number of elements in the second matrix along direction x.

In any of the above embodiments, for both the first and the second scans, blur is induced along the direction with the reduced number of elements.

Another way to provide accelerated 3D MRI, according to some embodiments of the present invention, is by acquiring an additional (third) MRI scan without reducing the number of elements along the z direction in the first and/or second acquisition matrices. In these embodiments the first MRI scan is acquired while employing phase-encoding along the x direction and also along one of the directions z and y, and while employing frequency-encoding along the other one of the directions z and y. Complementarily, the second MRI scan is acquired while employing phase-encoding along the y direction and also along one of the directions x and z, and while employing frequency-encoding along the other one of the directions x and z.

In embodiments in which it is desired to employ a third acquisition, the method can re-execute 11 following the execution of 14 to select a slice. Again, this operation can be skipped, unless operation 11 is executed before 12. The method can then proceed to 15, at which a third MRI scan is acquired by the MR scanner according to a third acquisition matrix, which is different from the first and the second acquisition matrices, wherein each of the first, second, and third acquisition matrices is a three-dimensional matrix.

The third MRI scan has a third type of MR image contrast. The third type of MR image contrast can be any of the aforementioned types of MR image contrast. The modalities of the MRI (presence of contrast agent, type of MR image contrast) are preferably different among operations 12, 14, and 15, as further detailed hereinabove. It is to be understood that while in the above description and in FIG. 1A the (optional) administration 13 of contrast agent is before the second acquisition 14, the present embodiments contemplate implementation in which contrast agent administration is executed after the second acquisition 14 and before the third acquisition 15. In this case, to ensure different modalities during the acquisitions, the first and second type of MR image contrast preferably differ, and the third type of MR image contrast can, but not necessarily, be the same as the first or the second types of MR image contrast.

Similarly to operations 12 and 14 above, the third MRI scan is also blurred. This is achieved by making the third acquisition matrix anisotropic. The direction or axes along which there are less matrix-elements in the third acquisition matrix is preferably complementary to the directions along which there are less matrix-elements in the other two acquisition matrices. Specifically, when the number of elements along direction x is less for the first matrix than for the other two matrices, and the number of elements along direction y is less for the second matrix than for the other two acquisition matrices, the number of elements along direction z is less for the third matrix than for the other two matrices. In this case, the third MRI scan is acquired while employing phase-encoding along direction z. An additional phase-encoding can also be employed along one of the directions x and y, and a frequency encoding can be employed along the other one of the directions x and y.

Preferably, in any of the acquisitions described above, the respective acquisition matrix has less elements along at least one of the directions for which phase-encoding is applied than along any readout direction.

Preferably, but not necessarily, the number of matrix-elements along any direction other than a direction along which the number of elements is minimal, is the same among the different matrices. For example, the number of elements of the first acquisition matrix along the y direction can be the same as the number of elements of the second acquisition matrix along the x direction.

While the embodiments above are described for cases in which the acquisitions of different MRI modalities are executed in series, wherein acquisitions of all slices are completed for a given MRI modality before beginning the acquisition of the first slice of the next MRI modality, this need not necessarily be the case, since, for some applications, it may be desired to perform interleaved acquisitions. For example, once a particular slice is selected, the method can acquire, for this selected set, two or more blurred MRI scans, wherein for each scan a different type of MR image contrast is selected.

When the grids over which the blurred MRI scans are k-space grids, the method continues to 16 at which a Fourier transform is applied to the data that represent the blurred MRI scans so as to convert the data from the spatial frequency domain of the k-space into the spatial domain of the image space. Typically a Fast Fourier Transform (FFT) is applied.

In some embodiments of the present invention the method proceeds to 17 at which the method co-register all the grids over which the blurred MRI scans are arranged. This can be done by any technique known in the art including, without limitation, at least one of: spatial co-registration, multimodal co-registration, atlas-based co-registration, longitudinal co-registration, motion correction, and deformable registration. In some cases, the MR scanner preforms automatic co-registration, in which case it is not necessary to execute operation 17. Operation 17 can be executed before or after operation 16.

The method proceeds to 18 at which a machine learning procedure that is trained to deblur MR images is fed with input data describing the blurred MRI scans.

As used herein the term “machine learning” refers to a procedure embodied as a computer program configured to induce patterns, regularities, or rules from previously collected data to develop an appropriate response to future data, or describe the data in some meaningful way.

Representative examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks (e.g., fully-connected neural network, convolutional neural network), instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis.

Preferably, the machine learning procedure comprises an artificial neural network.

Artificial neural networks are a class of algorithms based on a concept of inter-connected “neurons.” In a typical neural network, neurons contain data values, each of which affects the value of a connected neuron according to connections with pre-defined strengths, and whether the sum of connections to each particular neuron meets a pre-defined threshold. By determining proper connection strengths and threshold values (a process also referred to as training), a neural network can decode the range information from the input information (for example, the image data itself or some transform, e.g., a complex cepstrum transform, thereof). Oftentimes, these neurons are grouped into layers in order to make connections between groups more obvious and to each computation of values. Each layer of the network may have differing numbers of neurons, and these may or may not be related to particular qualities of the input data.

In one implementation, called a fully-connected neural network, each of the neurons in a particular layer is connected to and provides input value to those in the next layer. These input values are then summed and this sum compared to a bias, or threshold. If the value exceeds the threshold for a particular neuron, that neuron then holds a positive value which can be used as input to neurons in the next layer of neurons. This computation continues through the various layers of the neural network, until it reaches a final layer. At this point, the output of the neural network procedure can be read from the values in the final layer. Unlike fully-connected neural networks, convolutional neural networks operate by associating an array of values with each neuron, rather than a single value. The transformation of a neuron value for the subsequent layer is generalized from multiplication to convolution. In various exemplary embodiments of the invention the machine learning procedure is a convolutional neural network (CNN).

In some embodiments of the present invention the machine learning procedure is specific to the type of object being imaged. For example, when the object is a particular organ (e.g., brain, lungs, abdomen, limb, etc.) the machine learning procedure is a machine learning procedure that is trained specifically to deblur MR images of this particular organ, and when the object is a whole body of a human or animal, the machine learning procedure is a machine learning procedure that is trained specifically to deblur MR images of such whole body of human or animal.

In some embodiments of the present invention the machine learning procedure is a machine learning procedure that is trained specifically to deblur MR images acquired by an MR scanner of a particular type that is used for acquiring the blur MRI scans.

The input data that are fed to the machine learning procedure can be image-space data (i.e., after the application 17 of the Fourier transform), or k-space data (before the application of a Fourier transform). In the latter case, the Fourier transform 17 can be applied to the output data of the machine learning procedure, after this output data is received from the output of the machine learning procedure (operation 19, below).

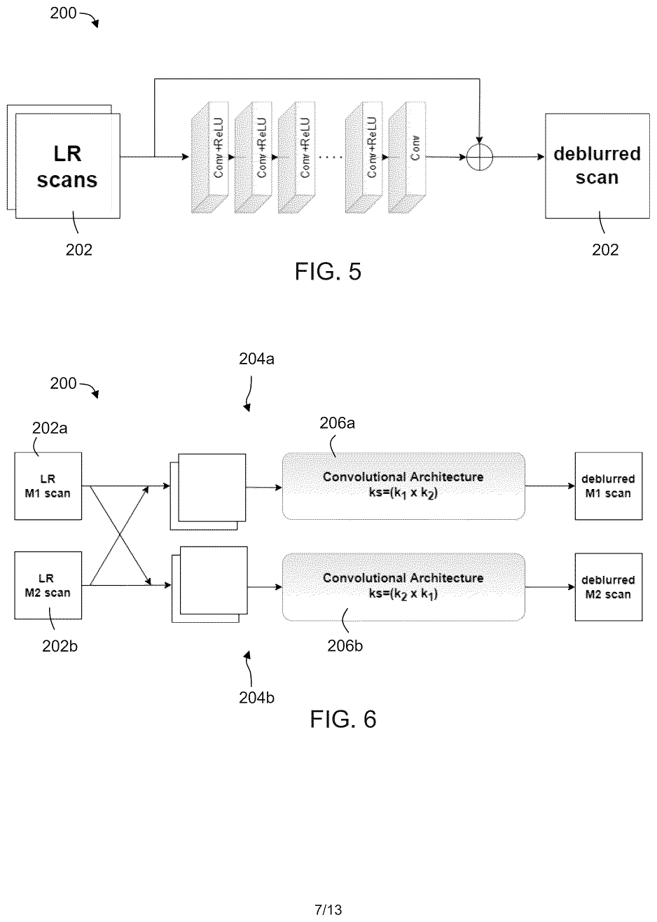

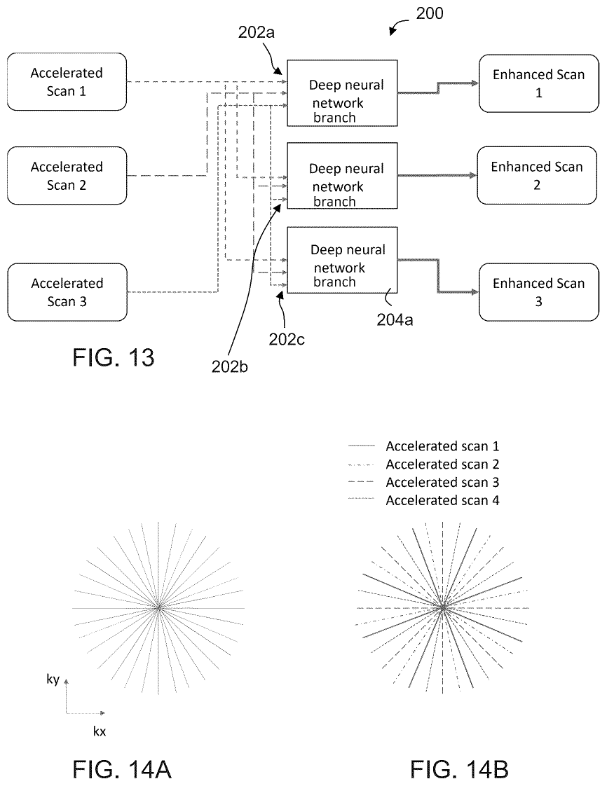

Representative examples of suitable architectures for a machine learning procedure 200 of the present embodiments are described in the Example section that follows (see FIGS. 5, 6, 12, 13). In some embodiments of the present invention the machine learning procedure 200 has a single branch with a single input layer 202 that receives all the input data (see FIGS. 5 and 12). In other embodiments, the machine learning procedure 200 has a plurality of branches 204a, 204b, with a respective input layer 202a, 202b, etc. for each branch (see FIGS. 6 and 13). Each of the input layers receives input data that describes one or the acquired blurred sets, but one or more of the hidden layers of each branch is connected to one or more of the layers (e.g., to the input layer) of the other branch. In some embodiments of the present invention machine learning procedure 200 is an artificial neural network, in which each of at least two of branches 204a has an anisotropic kernel 206a, 206b (FIG. 6) which is enlarged along a direction corresponding to an axis along which the MRI acceleration is executed.

The machine learning procedure can be trained according to some embodiments of the present invention by supervised training using a training dataset that can be prepared for the purpose of the training. Such a training dataset can include a plurality of MR images acquired without blurring and a respective plurality of blurred MRI scans, as further detailed hereinabove. The training dataset can be fed into a machine learning training program in a manner that each non-blurred image is used as a ground truth for a respective blurred image. Once the data are fed, the machine learning training program adjusts weights that are assigned to each component (e.g., neuron, layer, kernel, etc.) of the procedure, thus providing a trained machine learning procedure which can then be used without the need to re-train it.

When the machine learning procedure is specific to the type of object being imaged, the training dataset include MR images acquired without blurring and blurred MRI scans, where at least a majority, more preferably all the images in the dataset, are images of the respective type of object. When the machine learning procedure is a machine learning procedure that is trained specifically to deblur MR images acquired by an MR scanner of a particular type, the training dataset include MR images acquired without blurring and MR images constructed from the blurred sets, where at least a majority, more preferably all the images in the dataset, are acquired by the respective type of MR scanner. Examples of such training are provided in the Examples section that follows.

Referring back to FIG. 1A, the method proceeds to 19 at which output data corresponding to one or more deblurred MR image(s) is received from an output of the machine learning procedure 200. Preferably, the machine learning procedure 200 provides separate output for each part of the input data that corresponds to one of the blurred MRI scans. This provides deblurred MR image data for each of the MRI modalities that were employed while collecting the blurred MRI scans.

The method optionally and preferably proceeds to 21 at which one or more MR images are displayed on a display device.

The method ends at 21.

Reference is now made to FIG. 1B which is a flowchart diagram describing a method suitable for magnetic resonance imaging (MRI), in embodiments of the invention in which the acquisitions of MRI scans is over a radial grid, more preferably a radial k-space grid.

The method begins at 40 and optionally and preferably continues to 11 at which a slice of the object is selected, as further detailed hereinabove. As in the case of method 10 above, operation 11 is optional and in some embodiments of the present invention it is not executed.

The method continues to 42 at which a first MRI scan having a first type of MR image contrast is acquired by the MR scanner. The first type of MR image contrast can be any of the types of MR image contrast listed above with respect to method 10. The first MRI scan is acquired according to a first radial requisition protocol. With reference to FIG. 2C, this protocol comprises performing a scan along each of a plurality of radial sampling lines 52-1, 52-2, 52-3, etc. Preferably, each of these radial sampling lines 52 is a readout line, wherein as one moves along a particular line 52 each cell 32 of the grid 30 corresponds to a different bin of resonance frequencies for the same phase shift of the MR signal, and wherein different radial sampling lines 52 correspond to different phase shifts in the MR signal. Lines 52 thus form a first radial pattern 54 (solid lines) over the k-space. Preferably, the angular spacings 56 between adjacent lines 52 of pattern 54 are uniform over pattern 54.

In some embodiments of the present invention the method continues to 13 at which a detectable dose of an MR contrast agent is administered to the subject, as further detailed hereinabove.

Operation 13, when executed, can be executed before or after operations 11 and 42.

Following the execution of 42 and optionally following the execution of 13, the method optionally and preferably re-execute 11 to select a slice. This operation is only executed in cases in which operation 11 is executed before 42. Otherwise, this operation can be skipped.

At 44, a second MRI scan is acquired by the MR scanner. The second MRI scan is arranged over a grid of coordinates of the same type as the grid of coordinate over which the first MRI scan is arranged. In the present case, the grid is a radial grid.

The second image contrast can be any of the aforementioned types of MR image contrast.

The modalities of the first and second MRI scans preferably differ, as further detailed hereinabove with respect to the first and second MRI scans acquired by method 10. The second MRI scan is acquired according to a second radial requisition protocol. Referring to FIG. 2C, this second protocol comprises performing a scan along each of a plurality of radial sampling lines 62-1, 62-2, 62-3, etc. Similarly to lines 52, each of the radial sampling lines 62 is preferably a readout line, wherein as one moves along a particular line 62 each cell 32 of the grid 30 corresponds to a different bin of resonance frequencies for the same phase shift of the MR signal, and wherein different radial sampling lines 62 correspond to different phase shifts in the MR signal. Lines 62 thus form a second radial pattern 64 (dashed lines) over the k-space. Preferably, the angular spacings 66 between adjacent lines 62 of pattern 64 are uniform over pattern 64.

In various exemplary embodiments of the invention the number of lines in pattern 64 equals the number of lines in pattern 54. Preferably, the angular spacings 66 of pattern 64 equal the angular spacings 56 of pattern 54.

As illustrated in FIG. 2C, there is an angular offset 58 between patterns 54 and 64, so that the radial sampling lines 52 that describe the first radial acquisition protocol (and that belong to pattern 54) are interlaced with the radial sampling lines 62 that describe the second radial acquisition protocol (and that belong to pattern 64). Preferably, the interlacing forms a radial pattern 60 (encompassing both the dashed lines and the solid lines in FUG. 2C) having a uniform angular spacing 68 between adjacent radial sampling lines of pattern 60.

The first and second MRI scans acquired at 42 and 44 are referred to as “blurred” because each of angular spacings 56 and 66 is larger than the angular spacing 68, meaning that the phase-encoding density at each of these first and second MRI scans is less than the overall phase-encoding density of pattern 60. Thus, had pattern 60 been used for the acquisition of a single MRI scan there would have been more phase-encoding information in this single MRI scan than in any of the actually acquired MRI scans.

In some embodiments of the invention, the re-execute 11 following the execution of 44, so as to select a slice. Again, this operation can be skipped, unless operation 11 is executed before 42.

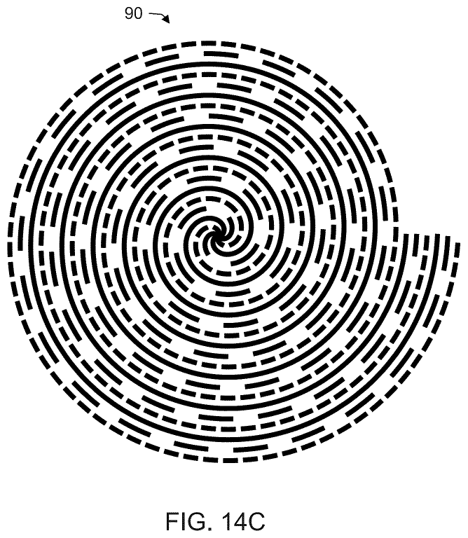

In some embodiments of the invention, the method proceeds to 45, at which one or more additional MRI scans are acquired by the MR scanner according to respective one or more additional radial protocol(s), optionally with appropriate slicing operations as further detailed hereinabove. The additional radial protocol(s) are similar to the first and second radial protocols, mutatis mutandis, and are therefore not specifically shown in FIG. 2C for clarity of presentation. Exemplified additional protocols are described in the examples section that follow (see FIG. 14B). Thus, each of the additional protocol comprises performing a scan along each of a plurality of radial sampling lines, where each of the radial sampling lines is preferably a readout line, with different radial sampling lines corresponding to different phase shifts in the MR signal. The lines of each of the additional protocols form a radial pattern over the k-space, preferably with uniform angular spacings between adjacent lines of each pattern, and preferably with the same number of lines in each pattern and/or the same angular spacings among different radial patterns.

Thus, when more than two MRI scans are acquired, there are also more that two radial patterns. Preferably, all the radial patterns are at angular offsets with respect to each other, and are therefore interlaced thereamongst. Preferably this interlacing forms an overall pattern in which the radial sampling lines of individual patterns form a cyclic sequence in the overall pattern. For example, when there are three individual patterns they are interlaced in a manner that as one completes a circle along the azimuthal direction ko, the second sampling line is always crossed after the first sampling line and before the third sampling line. A representative example of a cyclical interlacing of radial lines for the case of four patterns is illustrated in FIG. 14B.

When the grids over which the blurred MRI scans are k-space grids, the method continues to 16 at which a Fourier transform is applied as further detailed hereinabove. In some embodiments of the present invention the method proceeds to 17 at which the method co-register all the grids over which the blurred MRI scans are arranged, as further detailed hereinabove. The method then proceeds to 18 at which a machine learning procedure that is trained to deblur MR images is fed with input data describing the blurred MRI scans, to 19 at which output data corresponding to a one or more deblurred MR image(s) is received from an output of the machine learning procedure, and optionally to 21 at which one or more MR images are displayed on a display device.

The method ends at 46.

Reference is now made to FIG. 1C which is a flowchart diagram describing a method suitable for magnetic resonance imaging (MRI), in embodiments of the invention in which the acquisitions of MRI scans is over a spiral grid, more preferably a spiral k-space grid.

The method begins at 70 and optionally and preferably continues to 11 at which a slice of the object is selected, as further detailed hereinabove. As in the case of method 10 above, operation 11 is optional and in some embodiments of the present invention it is not executed.

The method continues to 72 at which a first MRI scan having a first type of MR image contrast is acquired by the MR scanner. The first type of MR image contrast can be any of the types of MR image contrast listed above with respect to method 10. The first MRI scan is acquired according to a first spiral requisition protocol. With reference to FIG. 2D, this protocol comprises performing a scan along each of a plurality of spiral sampling lines 82-1, 82-2, etc (only two spiral lines are illustrated for clarity of presentation, but the number of spiral lines can be larger than two). Preferably, each of these spiral sampling lines 82 is a readout line, wherein as one moves along a particular line 82 each cell 32 of the grid 30 corresponds to a different bin of resonance frequencies for the same phase shift of the MR signal, and wherein different spiral sampling lines 82 correspond to different phase shifts in the MR signal. Lines 82 thus form a first spiral pattern 84 (solid lines) over the k-space. Preferably, the radial spacings 86 between adjacent lines 82 of pattern 84 are uniform over pattern 84.

In some embodiments of the present invention the method continues to 13 at which a detectable dose of an MR contrast agent is administered to the subject, as further detailed hereinabove.

Operation 13, when executed, can be executed before or after operations 11 and 72.

Following the execution of 72 and optionally following the execution of 13, the method optionally and preferably re-execute 11 to select a slice. This operation is only executed in cases in which operation 11 is executed before 72. Otherwise, this operation can be skipped.

At 74, a second MRI scan is acquired by the MR scanner. The second MRI scan is arranged over a grid of coordinates of the same type as the grid of coordinate over which the first MRI scan is arranged. In the present case, the grid is a spiral grid.

The second image contrast can be any of the aforementioned types of MR image contrast. The modalities of the first and second MRI scans preferably differ, as further detailed hereinabove with respect to the first and second MRI scans acquired by method 10. The second MRI scan is acquired according to a second spiral requisition protocol. Referring to FIG. 2D, this second protocol comprises performing a scan along each of a plurality of spiral sampling lines 92-1, 92-2, etc. Similarly to lines 82, each of the spiral sampling lines 92 is preferably a readout line, wherein as one moves along a particular line 92 each cell 32 of the grid 30 corresponds to a different bin of resonance frequencies for the same phase shift of the MR signal, and wherein different spiral sampling lines 92 correspond to different phase shifts in the MR signal. Lines 92 thus form a second spiral pattern 94 (dashed lines) over the k-space. Preferably, the radial spacings 96 between adjacent lines 92 of pattern 94 are uniform over pattern 94.

In various exemplary embodiments of the invention the number of lines in pattern 94 equals the number of lines in pattern 84. Preferably, the angular spacings 96 of pattern 94 equal the angular spacings 86 of pattern 84.

As illustrated in FIG. 2D, the spiral sampling lines 82 that describe the first spiral acquisition protocol (and that belong to pattern 84) are interlaced with the spiral sampling lines 92 that describe the second spiral acquisition protocol (and that belong to pattern 94). Preferably, the interlacing forms a spiral pattern 60 (encompassing both the dashed lines and the solid lines in FIG. 2D) having a uniform angular spacing 98 between adjacent spiral sampling lines of pattern 90.

The first and second MRI scans acquired at 72 and 74 are referred to as “blurred” because each of radial spacings 86 and 96 is larger than the angular spacing 98, meaning that the phase-encoding density at each of these first and second MRI scans is less than the overall phase-encoding density of pattern 90. Thus, had pattern 90 been used for the acquisition of a single MRI scan there would have been more phase-encoding information in this single MRI scan than in any of the actually acquired MRI scans.

In some embodiments of the invention, the re-execute 11 following the execution of 74, so as to select a slice. Again, this operation can be skipped, unless operation 11 is executed before 72.

In some embodiments of the invention, the method proceeds to 75, at which one or more additional MRI scans are acquired by the MR scanner according to respective one or more additional spiral protocol(s), optionally with appropriate slicing operations as further detailed hereinabove. The additional spiral protocol(s) are similar to the first and second spiral protocols, mutatis mutandis, and are therefore not specifically shown in FIG. 2D for clarity of presentation. Each of the additional protocols comprises performing a scan along each of a plurality of spiral sampling lines, where each of the spiral sampling lines is preferably a readout line, with different spiral sampling lines corresponding to different phase shifts in the MR signal. The lines of each of the additional protocols form a spiral pattern over the k-space, preferably with uniform angular spacings between adjacent lines of each pattern, and preferably with the same number of lines in each pattern and/or the same angular spacings among different spiral patterns.

Thus, when more than two MRI scans are acquired, there are also more that two spiral patterns. Preferably, all the spiral patterns are interlaced thereamongst. Preferably this interlacing forms an overall pattern in which the spiral sampling lines of individual patterns form a cyclic sequence in the overall pattern. For example, when there are three individual patterns they are interlaced in a manner that as one moves along a radius of the spiral grid (e.g., direction kᵣ in FIG. 2D) the second sampling line is always crossed after the first sampling line and before the third sampling line, and so on. FIG. 14C illustrates a representative example an overall pattern 90 that is formed by cyclical interlacing of a first pattern (long dashed lines), a second pattern (short dashed lines) and a third pattern (solid lines).

When the grids over which the blurred MRI scans are k-space grids, the method continues to 16 at which a Fourier transform is applied as further detailed hereinabove. In some embodiments of the present invention the method proceeds to 17 at which the method co-register all the grids over which the blurred MRI scans are arranged, as further detailed hereinabove. The method then proceeds to 18 at which a machine learning procedure that is trained to deblur MR images is fed with input data describing the blurred MRI scans, to 19 at which output data corresponding to a one or more deblurred MR image(s) is received from an output of the machine learning procedure, and optionally to 21 at which one or more MR images are displayed on a display device.

The method ends at 76.

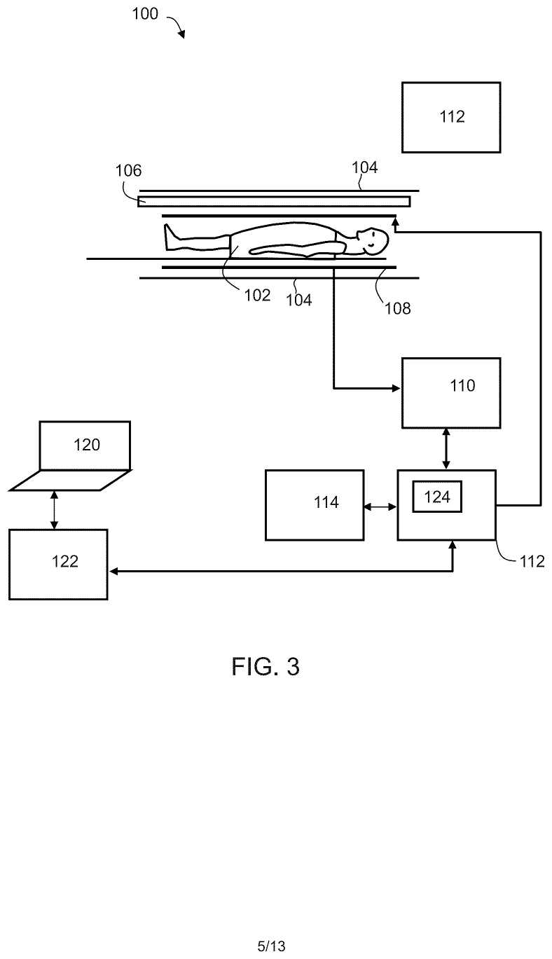

Reference is now made to FIG. 3 which is a schematic illustration of an MR scanner system 100 for imaging an object 102, according to some embodiments of the present invention. Object can be a full body of a human or an animal, or an organ thereof, or a non-living (e.g., artificial) object. System 100 comprises a static magnet system 104 which generates a substantially homogeneous and stationary magnetic field B₀ in the longitudinal direction, a gradient assembly 106 which generates instantaneous magnetic field gradient pulses to form a non-uniform superimposed magnetic field, and a radiofrequency transmitter system 108 which generates and transmits radiofrequency pulses to body 102.

System 100 further comprises an acquisition system 110 which acquires MRI scans from the body, and a control system 112 which is configured for implementing a pulse sequence. In particular, control system 112 is configured for allowing the operator to select between pulse sequences of different MRI modalities, as further detailed hereinabove. Control system 112 is also configured to control acquisition system 110 such that MRI scans are sequentially acquired.

In various exemplary embodiments of the invention system 100 further comprises an image producing system 114 which produces MR images from the signals. Image producing system 114 optionally and preferably implements a Fourier transform so as to transform k-space data into an array of image data.

The operation of system 100 is preferably controlled from an operator console 120 which can include a keyboard, control panel a display, and the like. Console 120 can include or it can communicate with a data processor 122.

Gradient pulses and/or radiofrequency pulses can be generated by a generator module 124 which is typically a part of control system 112. Generator module 124 produces data which indicates the timing, strength and shape of the radiofrequency pulses which are to be produced, and the timing of and length of the data acquisition window.

Gradient assembly 106 typically comprises Gₓ, G_{y} and G_{z} coils each producing the magnetic field gradients used for position encoding of the acquired signals. Radiofrequency transmitter system 108 is typically a resonator which is used both for transmitting the radiofrequency signals and for sensing the resulting MR signals radiated by the excited nuclei in object 102. The sensed MR signals can be demodulated, filtered, digitized etc. in acquisition system 110 or control system 112.

Data processor 122 is preferably configured for receiving the signals from control system 112, generate MR data, and execute selected operations of the method as further detailed hereinabove.

It is expected that during the life of a patent maturing from this application many relevant MR scanner will be developed and the scope of the term MR scanner is intended to include all such new technologies a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Complementary Phase-Encoding for Pair-Wise Neural Deblurring of Accelerated Brain MRI

This example describes a neural approach that jointly deblurs scan pairs acquired with mutually orthogonal phase-encoding directions. This leverages the complementarity of the respective phase encoded information as blur directions are also mutually orthogonal between the scans in the pair. This Example also described an architecture that is trained end-to-end and that is applied to T1W scan pairs consisting of one scan with contrast media injection (CMI), and one without CMI. Qualitative and quantitative validation is provided against state-of-the-art deblurring methods, for an acceleration factor of four beyond compressed sensing acceleration. The method described herein outperforms the compared methods.

### Introduction

The development of fast MRI acquisition techniques is useful because one of the considerations for MRI applicability in many clinical indications is the scanning time [14]. Besides poor patient experience, MRI scan duration also affects time to appointment, costs, and overall operational profitability [8].

In the classical process of MRI acquisition, raw data samples are used to fill the k-space. These are discrete data points placed on a finite grid in k-space. In this scheme, samples are acquired along the phase-encoding direction in a line-by-line ordering, with acquisition duration affected by the phase-encoding rather than the frequency encoding (readout) direction [4].

Over the past decade, compressed sensing (CS) techniques have been successfully applied to MRI data acquisition, leading to significant scanning time reductions [19, 20]. CS is a mathematical framework developed for data reconstruction from highly under-sampled data by leveraging sparse representations. In CS-MRI, the image is recovered from semi-random incomplete sampling of k-space data using an appropriate nonlinear recovery scheme.

Recently, deep convolutional neural networks have achieved superior results in generic computer vision tasks, such as segmentation, registration, image deblurring and denoising [9, 16, 21, 27, 29, 30, 32].

In a fully convolutional network with anisotropic kernels was proposed. The network was optimized for the task of deblurring low resolution (LR) brain MRI using a perceptual cost

function. A GAN based architecture was suggested in for the task of MRI reconstruction. The mapping between the low and high resolution images is learnt using a generator with both pixel-wise and perceptual loss components on its output. Another loss, in the frequency domain, is also computed, after applying FFT on the input and the generator's output.

Cross-modal brain MRI enhancement is reported in [27, 29, 32], where one of the input modalities is enhanced based on the other. Tsiligianni [27] proposes a convolutional network that uses a high resolution (HR) scan of one (source) modality to super-resolve an image of a different (target) modality. Xiang et al [29] present a Unet-based architecture that uses HR T1W scans to reconstruct under-sampled T2W scans. Zhou et al [32] also use T1W as prior to reconstruct T2W scans. They use a recurrent network where each block is comprised of both k-space restoration network and image restoration network.

Iwamoto et al [9] use synthesized scans, down-sample them to create blurred versions, and use a high quality scan in another modality to learn deblurring. The resulting network is used to enhance an LR scan in one modality using an HR guidance scan in another modality.

Different from natural image processing studies, where many benchmark datasets are available [2, 22, 31], the medical imaging field is short of such resources [12].

Creating medical image datasets requires resources such as scanners, informed consent by patients and various regulatory approvals. Another important observation regarding medical image enhancement is that many works are demonstrated only on synthesized data [9, 14, 24, 27]. The synthesis is carried out by simulating sub-sampling of a fully sampled k-space data. The inventors found that such datasets are ofentimes not accurately resemble real LR data. Recently, a dataset of real LR and HR paired scans has appeared [1]. Some works on MRI acceleration use data that was not acquired using CS protocols. The inventor found that this makes performance evaluation difficult, since the acceleration comes at the expense of potential acceleration by the use of CS [21].

MRI deblurring is closely related to natural image enhancement problems such as super-resolution [17, 23] and deblurring [16]. They resemble in their definition as inverse problems and in the applicable deep learning architectures, providing an additional comparison basis to SOTA methods [16]. Kupyn et al [16] present a GAN architecture that uses a conditional GAN with a perceptual loss incorporated in its loss function components. This is a follow up work of [15] reaching SOTA results. One difference between medical and natural image enhancement is that while in the latter a photo-realistic eye-pleasing result is usually sought, in medical image analysis details must be correctly restored to preserve diagnostic value.

This Example describes an end-to-end framework for deblurring accelerated multi-modal MRI scans. An advantage of the technique of the present embodiments is that it provides a joint deblurring scheme for Low Resolution (LR) MRI scans of different modalities. Another advantage of the technique of the present embodiments is that it provides better deblurring results in comparison to single-modality methods.

The technique described in this example creates and uses a patient dataset where each entry in the dataset consists of four brain MRI scans of the same patient: two pairs of LR and HR scans, one pair with a contrast media injection (CMI) and one without CMI, where the phase-encoding direction is changed between the two LR scans.

### Methodology

### Registration

Image registration is the alignment of two or more images into the same coordinate system. The image transformed into another coordinate system is referred to below as the moving image and the image remaining in its original coordinate system is referred to below as the fixed image. Image registration is employed in many medical imaging applications, as it helps to establish correspondence between scans taken at different times, of different subjects, with different modalities. In this example, which is based on actual patients' scans, registration is a pre-processing step consisting of several elements. Firstly, a 3D rigid-body registration between each LR-HR pair is performed. Unlike researches based on synthetic data, movements of the patient between the two scans are also addressed, otherwise the accuracy of LR-HR pairing may be reduced and may lead to inferior deblurring results. Next, registration between the different modalities takes place. The 3D rigid-body registration between the two modalities is calculated and applied to all registered scans of the moving image modality. At this point, all four (two LR, two HR) scans acquired from a single patient are registered. A normalization process is applied a to align all four scans together and to eventually have the entire dataset aligned together. The registration is implemented via the SimpleElastix framework [13, 25].

Brain atlases are useful in many medical applications. Brain MRI atlases are a collection of MRI scans collected from several different individuals. These scans are averaged while incorporating statistical knowledge in a voxel-wise, regional, or global manner. In this example registration to atlas was added, as it allows for more consistency within the entire dataset and not only scan-wise. As brains of several different subjects were registered, 3D anisotropic scaling and 3D rigid transformation was calculated. In this Example, only the parameters of the rigid transformation were applied. The reason is that while registering, interpolation is applied so it is desired to avoid unnecessary deformation to the signal. The machine learning procedure that was employed in this example was an artificial neural network that was trained on images registered to atlas. Once the deblurred images were obtained the inverse transformation was applied to return into the original image space.

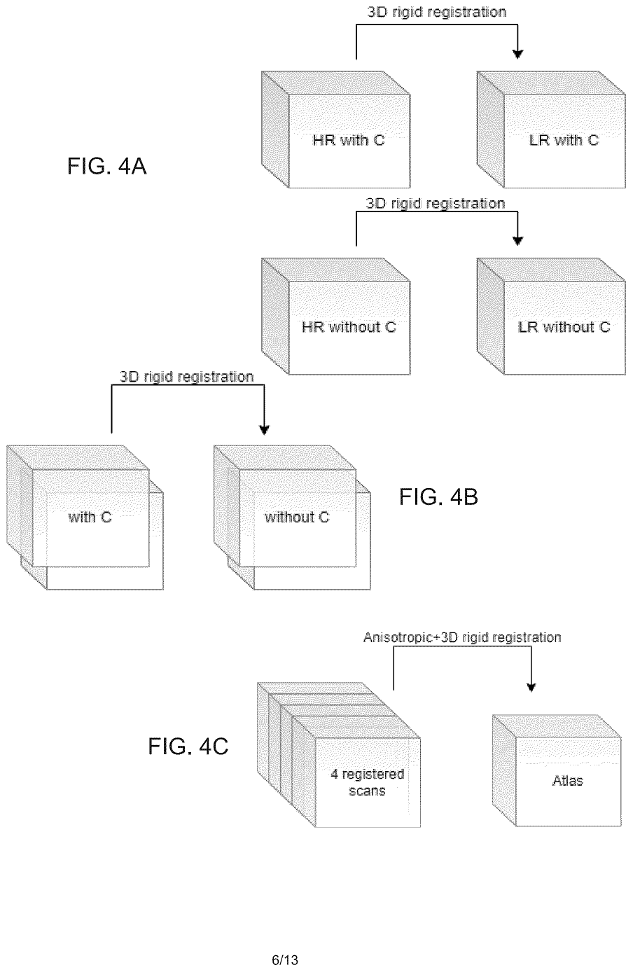

The atlas used in this example is composed of unbiased averages of 152 T1-weighted MRI scans from the ICBM study [3, 5, 6]. The registration process is visualized in FIGS. 4A-C, illustrating 3D registration to the atlas. FIG. 4A illustrates registration of HR scan to LR scan, FIG. 4B illustrates registration of contrasted scans to non-contrasted scans, and FIG. 4C illustrates registration of all four registered scans to the atlas.

### Architecture

The machine learning procedure used in this example is an artificial neural network which in this example is a fully convolutional neural network, and which is referred to below as a Multi-Modal MRI Deblurring Network (MMMD-Net). The network receives, as input data, two LR MRI scans of two different modalities, each blurred in a different axial direction (x or y) and outputs a deblurred version of both images. The network contains two branches. FIG. 5 illustrates the branch architecture. Its input is two registered scans of the two different modalities, stacked one on top of the other. It outputs a deblurred version of the top input scan. In this example, the branch architecture was fully convolutional architecture with 9 convolution layers, 64 filters followed by a ReLU activation function in each layer. The architecture illustrated in FIG. 5 can also be used as a non-branched neural network, with an input layer 202 and an output layer 208 that provide image data of a deblurred scan.

FIG. 6 illustrates the architecture of the MMMD-Net. Anisotropic complementary kernels are used for the two branches, where the kernel size ks is specified by its height and width, ks=(kh x kw). The upper branch outputs a deblurred version of the T1W scan with ks=(3×7) and the lower branch outputs a deblurred version of the T1W CMI scan with ks=(7×3). The anisotropic kernels were selected to be enlarged in the phase-encoding direction, so as to take advantage of the prior knowledge regarding the blur direction [21].

### Residual Learning

Residual Learning [7] is useful in various image enhancement problems such as deblurring, super-resolution and denoising.

With residual learning the network learns the difference between the restored and original images rather than learning the restored image itself. For image enhancement tasks learning the difference is desirable as it promotes similarity between the input and the output images.

ŷ=F(x;θ)+x (EQ. 1)

Here, ŷ is the reconstructed image, x is the input (in this example the LR image), and F(x; θ) is a function that represents the learned residual mapping.

### Loss Function

The network is trained using a multi-component loss function.

L=L_{perceptual}+αL_{SSIM} (EQ. 2)

Pixel-wise loss, such as L1 or MSE, can be extremely sensitive to small pixel shifts, making it very different from the perception of the human reader comparing the deblurred output image to the ground truth (GT) image. Perceptual loss used in image transformation tasks [10] is based on the differences between high-level features of the image, rather than between pixel values. Pre-trained convolutional neural networks are often used in order to extract these high-level features.

In the present example, a pre-trained VGG-16 network was used as a feature extractor. Both the HR image and the output image are fed into the VGG model, where features are extracted from multiple layers. The L1 distance is calculated between each pair of extracted features. Eventually the mean is taken to form the final L_{perceptual} loss component.

$${L}_{perceptual} = \frac{1}{\vert I \vert} {\sum }_{i ∈ I} {‖ {ϕ}_{i} ( y ) - {ϕ}_{i} ( \hat{y} ( x ; θ ) ) ‖}_{1} \qquad ( EQ . 3 )$$

Here φᵢ is feature extracted from the ith VGG-16 layer and I=[0, 4, 9, 23, 26, 30]. Layer 0 refers to the VGG-16 input, which in the present Example is the HR and deblurred output images. Its contribution to the sum is in fact the pixel-wise L1 distance between the HR and deblurred output images. The other layers are numbered according to the sequential model numbering in the Torchvision pre-trained VGG-16 model.

SSIM (Structural Similarity index) [28] is often used for measuring the similarity between two images. It allows for quality comparison between two images where one of them is considered to be of superior quality while the other aspires to match its quality. SSIM, similarly to perceptual loss, is used to analyze the difference between features extracted from the images. The index makes use of three image features: structure, contrast and luminance.

$$SSIM ( x , y ) = \frac{( 2 {μ}_{x} {μ}_{y} + {c}_{1} ) ( 2 {σ}_{xy} + {c}_{2} )}{( {μ}_{x}^{2} + {μ}_{y}^{2} + {c}_{1} ) ( {σ}_{x}^{2} + {σ}_{y}^{2} + {c}_{2} )} \qquad ( EQ . 4 )$$

As it is desired to maximize similarity between the network output and the HR image, corresponding to a high SSIM value, L_{SSIM} is set to be L_{SSIM}=−SSIM and is multiplied by a positive factor α.

The loss expression (EQ. 2) is calculated per branch, and the network's total loss is the sum of the two.

### Experiments

### Dataset

A dataset was assembled with brain MRI scans collected from 9 distinct subjects, with different medical conditions. Prior to the scanning, each patient provided a written consent in accordance with the IRB clearance delivered by our institution. Each subject provided 4 scans, all acquired using CS protocols [19, 20]. In this dataset, multi-modality MRI is the acquisition of two scan types, T1W scans and T1W CMI-enhanced scans. Both brain scans are commonly acquired in diagnostic practice. Thus, the following scans were acquired: Two HR scans, with acquisition matrix (2D Cartesian k-space sampling) size of 240×240, and two complementary LR sagittal scans. One LR sagittal scans was a T1W scan with a 240×60 acquisition matrix, resulting from reduction by a factor of 4 in the phase-encoding direction. This reduction translates into an acceleration factor of 4. In the image domain, the acceleration leads to strong blur artifacts along the x axis. The other LR sagittal scan was a T1W CMI-enhanced scan with a 60×240 acquisition matrix and the same acceleration factor. This time, the phase-encoding direction is swapped, such that in the image domain, the acceleration leads to strong blur artifacts along the y axis.

The scanner firmware presented the reconstructed HR and LR scans as 512×512×170 voxel sets.

### Training

All the experiments were conducted with a NVIDIA Geforce RTX 3070 GPU. PyTorch was used for the implementation. The network was trained for 150 epochs with a batch size of 6. The training duration was 12.5 hours. Adam optimizer [11] was used, with betas=0.9, 0.99 and weight decay=0.001. The initial learning rate was set to 0.001 and a SGDR [18] scheduler was used with minimum lr=0.0001, 5 iterations for the first restart and a multiplication factor of 2 for the time between restarts.

### Results

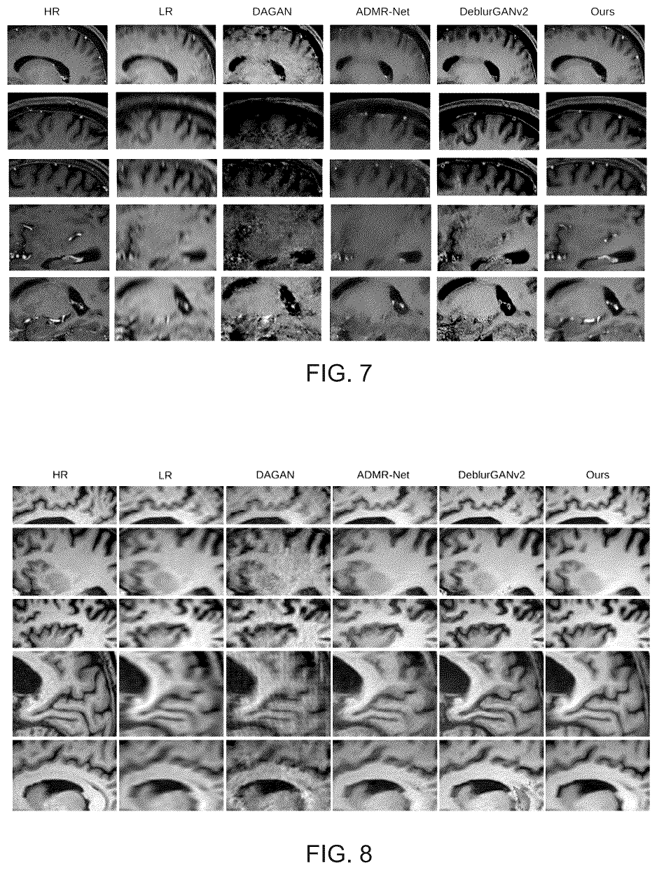

For evaluating the network in comparison to conventional methods, it was trained, along with ADMR-Net [21], DeblurGANv2 [16] and DAGAN [30], on the dataset prepared as described herein using a leave-one-out validation strategy. The configurations used for training the SOTA methods matched those presented in their original papers. Training times for these algorithms were respectively 117% 102% and 167% compared to the method of the present embodiments. Visual results are presented per modality in FIG. 7 (T1W with CMI) and 8 (T1W without CMI). Patches are presented to allow for the observation of details. In both FIGS. 7 and 8, a strong blur is observed in the LR column. FIG. 7 demonstrates that unlike the method of the present embodiments, all conventional methods struggled to restore details originating from CMI. The images generated by DAGAN demonstrate no such details. ADMR-Net has mostly failed to restore them as well. In DeblurGAN the attempt to restore the CMI-contrast details resulted in strong artifacts. In contrast, the MMMD-Net described was able to restore CMI-contrast details in different areas of the brain. In addition to preservation of CMI-contrast details, the method of the present embodiments was able to restore fine structural details of the brain and to achieve superior results in this aspect as well. FIG. 8 also demonstrates the superior performance of the method of the present embodiments. With DAGAN the results are still blurred and have strong patchy looking artifacts. ADMR-Net provides a rather blurry result as well. DeblurGAN produced artifacts in some of the patches along with blurred regions especially noticeable in detailed small regions.

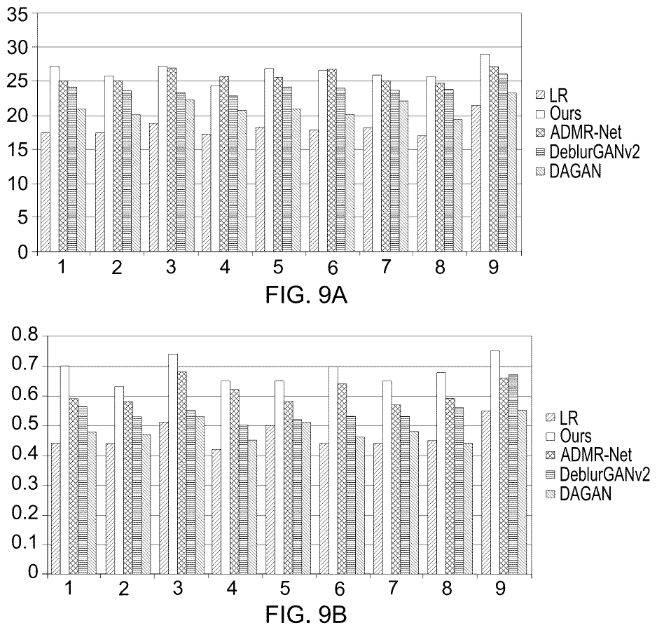

Quantitative performance comparisons in terms of the peak signal-to-noise ratio (PSNR) and SSIM indices are illustrated graphically in FIGS. 9A-B and 10A-B and are summarized in Tables 1 and 2, below. In Tables 1 and 2, the results obtained for the method of the present embodiments are denoted “This work.”

TABLE 1
PSNR mean and STD values per method per modality

| Modality | LR | DAGAN | ADMR-Net | DeblurGANv2 | This work |
|---|---|---|---|---|---|
| T₁W | 16.33 ± 0.6 | 16.88 ± 0.4 | 24.04 ± 0.81 | 23.2 ± 1.12 | 24.76 ± 1 |
| T₁W with CMI | 18.15 ± 1.27 | 21.01 ± 1.13 | 25.69 ± 0.87 | 23.91 ± 0.83 | 26.41 ± 1.22 |

TABLE 2
SSIM mean and STD values per method per modality

| Modality | LR | DAGAN | ADMR-Net | DeblurGANv2 | This work |
|---|---|---|---|---|---|
| T₁W | 0.5 ± 0.03 | 0.44 ± 0.02 | 0.58 ± 0.03 | 0.55 ± 0.04 | 0.63 ± 0.04 |
| T₁W with CMI | 0.46 ± 0.04 | 0.48 ± 0.03 | 0.61 ± 0.04 | 0.55 ± 0.058 | 0.68 ± 0.04 |

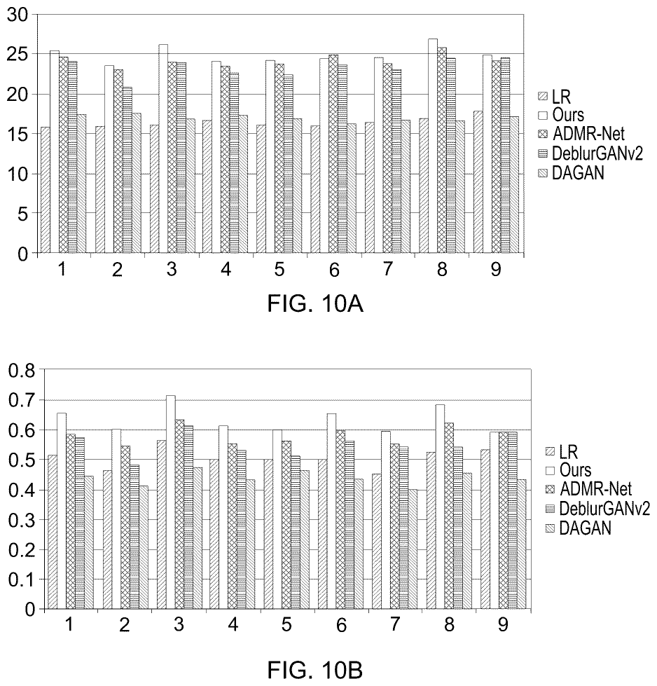

The graphs in FIGS. 9A-B and 10A-B are presented per modality and per performance index. Specifically, FIG. 9A shows PSNR index in T1W MRI scans with CMI, FIG. 9B shows SSIM index in T1W MRI scans with CMI, FIG. 10A shows PSNR index in T1W scans, and FIG. 10B shows SSIM index in T1W scans. Each of FIGS. 9A-10B shows nine bar quintets. In each quintet, the leftmost bar corresponds to the results for LR, the second from left bar corresponds to the method of the present embodiments, the next bar corresponds to ADMR-Net, the next bar corresponds to DeblurGANv2, and the fifth bar corresponds to DAGAN. In agreement with the comparative visual results, the method of the present embodiments scored the highest PSNR and SSIM values for both modalities compared to all other methods. The LR images obtained the lowest scores in all but one case.

### Conclusions

This example presented a fully convolutional neural network for jointly deblurring multimodal brain MRI scans. The network was applied to multi-modal brain scans, where the modalities are T1W with and without CMI. The scans were acquired with phase-encoding directions orthogonal between the two modalities, and with an acceleration factor of 4 along the phase-encoding directions. For training and validation, a unique dataset of actual T1W brain scans with various medical conditions was assembled, with four scans per subject: HR and LR, with and without CMI where the phase-encoding direction is switched. Experimental results reveal superior deblurring performance with respect to conventional single-modality methods, both visually and in terms of the PSNR and SSIM indices. These results demonstrate substantial MRI acceleration, allowing higher MRI throughput, lower costs and better accessibility to MRI.

### Example 2

### Three-Dimensional MRI and Non-Cartesian Grid

MRI images can be acquired in a two-dimensional slice-by-slice manner, or alternatively using three-dimensional (3D) acquisition techniques that allow volumetric imaging of an entire region of interest in a single scan. With 3D MRI acquisition, thin contiguous slices can be retrospectively reconstructed in any orientation. This improves spatial resolution and reduces partial volume effects compared to conventional 2D scanning. 3D MRI data can also be reformatted for multi-planar analyses or projections. Advantages of 3D MRI acquisition include higher signal-to-noise ratios, improved spatial resolution, and reduced scanning times. It is recognized that 3D MRI is more demanding on time consumption.

For 3D imaging, phase-encoding is applied along two orthogonal directions (e.g., x and y), while frequency encoding is applied in the third (e.g., z) direction. This allows for volumetric scanning of the entire region of interest.

T_{ACQ}=NEX*Nₓ*N_{y}*TR

Where x and y are the phase-encoding direction and hence Nₓ and N_{y} are the number of points in the phase-encoding matrix. NEX is the number of excitations upon the signal being averaged. TR is the repetition time between two successive pulses.

The total scan time is proportional to the resolution in each direction as well as the TR. 3D MRI acquires the entire volume in a single shot, enabling high-resolution isotropic imaging. It is appreciated, however, that the long acquisition time can lead to greater motion artifacts and patient discomfort.

### 3D Triplet Accelerated Scan

The primary factor determining 3D MRI acquisition time is the number of phase-encoding steps Nₓ and N_{y}, which set the spatial resolution. Reducing either Nₓ or N_{y} reduces the total scan duration. However, simply decreasing Nₓ or N_{y} using standard sampling leads to under-sampling artifacts in the reconstructed images, including aliasing and reduced signal-to-noise ratio.

To mitigate this, specialized under-sampling techniques can be employed. Conventional parallel imaging methods like GRAPPA and SENSE allow for reducing phase-encoding steps by taking advantage of multi-coil spatial information. Conventional compressed sensing techniques exploits sparsity and successfully reconstructs MRI from randomly under-sampled k-space data.

These advanced sampling techniques make it possible to acquire high-quality 3D MRI data with fewer phase-encoding steps and faster scan times. However, the inventors found that the reconstruction process becomes more complex and computationally demanding. The inventors also found that there is also a limit to how much a phase matrix can be reduced before image quality becomes unacceptable.

This Example describes a scheme optimized for multi-sequence 3D MRI acquisition protocols (multiple 3D scans with different contrast properties acquired during an imaging session). This scheme can be used in combination with the acceleration technique, e.g. compressed sensing. The technique described herein allows under-sampled acquisition that reduces the scan time by decrementing the phase-encoding steps in alternating directions for successive contrasts.

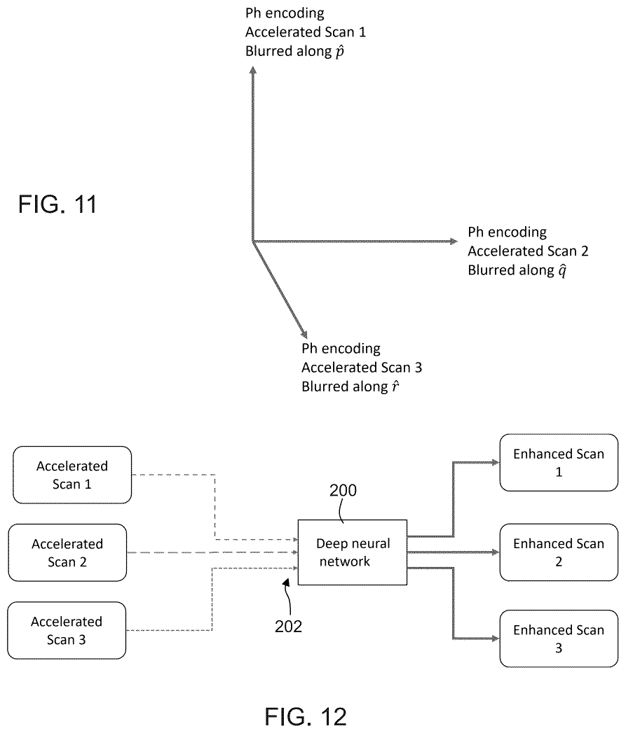

Consider, for example, the acquisition of three co-registered 3D MRI scans with different contrasts (e.g., T1-weighted, T2-weighted, and T1-FLAIR), where each scan is acquired in k-space defined by common orthogonal directions \hat{p}, \hat{q}, \hat{r}. The acquisition matrix size for each scan is set, according to some embodiments of the present invention, as follows (see also FIG. 11):

$$\frac{P}{f} \cdot Q \cdot R$$

$$P \cdot \frac{Q}{f} \cdot R$$

$$P \cdot Q \cdot \frac{R}{f}$$

where P, Q, and R are the number of k-space samples acquired along each direction \hat{p}, \hat{q}, \hat{r}, respectively, for a reference (not acquired in practice) full-resolution scan.

Each of the 3 accelerated scans is accelerated along a different phase-encoding directions (\hat{p}, \hat{q}, \hat{r}). Acceleration is achieved by reducing the number of k-space points (the aforementioned Ni number, i=x,y,z) acquired along the phase-encoding direction by a factor of f. This reduction in k-space sampling results in blurring along the accelerated direction, which is different in each one of the 3 scans. Conversely, each one of the said scans provides high-resolution, that is non-blurred, information along the 2 other mutually orthogonal directions that are not accelerated.

The acquired blurred images constitute a multi-channel 3D volume with channels corresponding to each of the acquired contrasts (e.g., T1-weighted, T2-weighted, and T1-FLAIR). Since blur is along a different direction in each acquired scan, this multi-channel input contains complementary information from the different scan contrasts which can be leveraged by the deblurring neural network. Two examples of deblurring architectures are considered in this example. These include (i) a single neural network with multiple output channels corresponding to each input contrast, which is optimized together through shared weights (FIG. 12), and (ii) a multi-branch neural network with separate branches for each contrast and hence separate weights (FIG. 13).

The technique can be used also when there are two MRI scans (e.g., a T1-weighted MRI scan and a T2-weighted MRI scan, or a T1-weighted MRI scan and a T1-FLAIR MRI scan). In this case, acceleration can be applied along any two of the three orthogonal k-space directions \hat{p}, \hat{q}, \hat{r}. For example, acceleration may be applied along \hat{p} and \hat{q}, along \hat{p} and \hat{r}, along \hat{q} and \hat{r}. All combinations and permutations of accelerating two directions for the two scan acquisition schemes can be employed. This provides flexibility to choose which two directions to accelerate based on the orientation of the anatomy and application. The two directions along which acceleration is applied can be chosen to minimize artifacts for the target anatomy.

The acceleration factor f can be set independently for each accelerated scan direction corresponding to \hat{p}, \hat{q}, \hat{r}. For example, when two MRI scans are acquired with acceleration along \hat{p} and \hat{q}, the acceleration factor, f_{q}, along the direction p can be different from the acceleration factor, f_{q}, along the direction \hat{q} (e.g., f_{q}=4, f_{q}=3). This allows asymmetric acceleration, with more aggressive acceleration along one direction than along the other. Unequal acceleration factors provide flexibility to balance image resolution, acquisition time, and artifacts in each direction.

It is appreciated that the basic architectures shown in FIGS. 12 and 13 can be customized as needed for the specific application, for example, by changing the deep neural network block.

A perceptual loss can be embedded in the network (see EQ. 5, below) with pre-trained weights from a model that used the same MRI sequences, the weights of the pre-trained feature extraction network are held fixed, while the MRI reconstruction network is trained to minimize the perceptual loss. This guides the network to produce reconstructions that reside in a similar feature space to the fully sampled reference images. The perceptual loss provides a supplementary training signal alongside pixel-wise losses, resulting in images that appear more realistic to human visual perception.

Lₚ=∥φₗ(xᵣ)−φₗ(xₜ)∥₂ (EQ. 5)

where, Lₚ is the perceptual loss, φₗ is layer l of the pretrained feature extraction network φ, xᵣ is the reconstructed image, and xₜ is the target fully-sampled image.

### Non-Cartesian Grid of Acquisition

The concept described above for accelerating cartesian k-space sampling scheme in 2 or 3D can also be utilized to non-cartesian acquisition schemes, such as, but not limited to, radial k-space sampling or spiral k-space sampling.

For example in a conventional radial sampling scheme, the k-space is sampled sequentially by a set of straight lines, each passing through the k-space origin with a different slope (FIG. 14A). For MRI scan acquisition along N radial sampling lines, a uniform angular distribution of the k-space radial sampling lines result in an angular shift of 360/N degrees between adjacent radial sampling lines.

According to some embodiments of the present invention n≥2 accelerated MRI scans are acquired. The MRI scans are “accelerated” because they contain less radial sampling lines then they would have contained, had a single MRI scan was acquired. Specifically, each MRI scan is acquired along N/n radial sampling lines, with an equal angular shift between each radius of the same MRI scan equal to 360/(n·N) degrees. This provides n sets of N/n radial sampling lines. The radial sampling lines of the sets are angularly-shifted from each other in the k-space in order to obtain a sampling pattern of N radial sampling lines, with an angular shift of 360/N degrees between adjacent radial sampling lines, except that the radial sampling lines of different MRI scan are interlaced thereamongst, as illustrated in FIG. 14B.

According to some embodiments of the present invention a set of N/4 radial lines sample the k-space radially for each MRI scan, with an equal angular shift between each radius of the same set equal to 360/(4N) degrees. The 4 sets of radial sampling lines are angularly-shifted from each other in the k-space in order to obtain a sampling pattern of N radial sampling lines, with an angular shift of 360/N degrees between adjacent radial sampling lines, except that the radial sampling lines of different MRI scan are interlaced thereamongst, as illustrated in FIG. 14B.

Consider acquisition of, e.g., n≥2 four accelerated MRI scans (e.g., T1W, T2W, T1FLAIR, T2FLAIR) using a radial scheme. According to some embodiments of the present invention a set of N/4 radial lines sample the k-space radially for each MRI scan, with an equal angular shift between each radius of the same set equal to 360/(4N) degrees. The 4 sets of radial sampling lines are angularly-shifted from each other in the k-space in order to obtain a sampling pattern of N radial sampling lines, with an angular shift of 360/N degrees between adjacent radial sampling lines, except that the radial sampling lines of different MRI scan are interlaced thereamongst, as illustrated in FIG. 14B.

In the sampling pattern, consecutive radial sampling lines are associated to a cyclic sequence along the azimuthal direction of the four MRI scans, e.g., T1W-T2W-T1 FLAIR-T2 FLAIR-T1W-T2W-T1 FLAIR-T2 FLAIR- . . . -T1W-T2W-T1 FLAIR-T2 FLAIR . . . .

The situation is similar in the case of spiral MR acquisition, except that instead of a plurality of interlaced radial sampling lines shown in FIG. 14B, there is a plurality of interlaced spiral sampling lines shown in FIG. 14C. The illustration in FIG. 14C corresponds to three MRI scans of different modalities. For example, when the three MRI scans are a T1W MRI scan, a T2W MRI scan and a T1 FLAIR MRI scan, the sampling pattern 90, includes consecutive spiral sampling lines forming a cyclic sequence along the radial direction, e.g., T1W-T2W-T1 FLAIR-T1W-T2W-T1 FLAIR- . . . -T1W-T2W-T1 FLAIR and so on.

In any of these non-cartesian cases, the resulting accelerated scans can be jointly processed using neural networks as described herein to obtain enhanced scans. Note that the de-blurring can be performed either by applying the networks to the accelerated scans after transformation (by Fourier transform) to the image space as was done implicitly for the cartesian case, or, by applying the networks directly in the k-space, before the projection onto the image space by the Fourier transform.

It is appreciated that the above is applicable for any number of MRI scans, and also to other non-cartesian grids.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

[1] Accelmr 2020 prediction challenge (2020), www(dot)caccelmrorg(dot)wordpress(dot)com/
[2] Bevilacqua, M., Roumy, A., Guillemot, C., line Alberi Morel, M.: Low-complexity single-image super-resolution based on nonnegative neighbor embedding. In: Proceedings of the British Machine Vision Conference. pp. 135.1-135.10. BMVA Press (2012) www(DOT)doi(DOT)org/10.5244/C.26.135
[3] Collins, D. L., Zijdenbos, A. P., Baar'e, W. F. C., Evans, A. C.: Animal+insect: Improved cortical structure segmentation. In: Kuba, A., S ̌a'amal, M., Todd-Pokropek, A. (eds.) Information Processing in Medical Imaging. pp. 210-223. Springer Berlin Heidelberg, Berlin, Heidelberg (1999)
[4] Edelstein, W. A., Hutchison, J. M. S., Johnson, G., Redpath, T.: Spin warp NMR imaging and applications to human whole-body imaging. Physics in Medicine and Biology 25(4), 751-756 (July 1980).
   www(DOT)doi(DOT)org/10.1088/0031-9155/25/4/017
[5] Fonov, V., Evans, A. C., Botteron, K., Almli, C. R., Mckinstry, R. C., Collins, D. L.: Unbiased average age-appropriate atlases for pediatric studies. NeuroImage 54(1), 313-327 (2011).
   www(DOT)doi(DOT)org/10.1016/j.neuroimage.2010.07.033,
   www(dot)www(dot)sciencedirect(dot)com/science/article/pii/S1053811910010062
[6] Fonov, V., Evans, A., Mckinstry, R., Almli, C., Collins, D.: Unbiased nonlinear average age-appropriate brain templates from birth to adulthood. NeuroImage 47, S102 (2009). www(DOT)doi(DOT)org/10.1016/S1053-8119(09)70884-5, organization for Human Brain Mapping 2009 Annual Meeting
[7] He, K., Zhang, X., Ren, S., Sun, J.: Deep residual learning for image recognition (2015).
   www(DOT)doi(DOT)org/10.48550/ARXIV.1512.03385,
   www(DOT)arxiv(DOT)org/abs/1512.03385
[8] Hollingsworth, K. G.: Reducing acquisition time in clinical mri by data undersampling and compressed sensing reconstruction. Phys. Med. Biol. 60(21), 297-322 (2015)
[9] Iwamoto, Y., Takeda, K., Li, Y., Shiino, A., Chen, Y. W.: Unsupervised mri super-resolution using deep external learning and guided residual dense network with multimodal image priors (2020)
[10] Johnson, J., Alahi, A., Fei-Fei, L.: Perceptual losses for real-time style transfer and super-resolution (2016).
   www(DOT)doi(DOT)org/10.48550/ARXIV.1603.08155,
   www(DOT)arxiv(DOT)org/abs/1603.08155
[11] Kingma, D. P., Ba, J.: Adam: A method for stochastic optimization (2014).
   www(DOT)doi(DOT)org/10.48550/ARXIV.1412.6980,
   www(DOT)arxiv(DOT)org/abs/1412.6980
[12] Kiryati, N., Landau, Y.: Dataset growth in medical image analysis research. Journal of Imaging 7, 155 (August 2021).
   www(DOT)doi(DOT)org/10.3390/jimaging7080155
[13] Klein*, S., Staring*, M., Murphy, K., Viergever, M. A., Pluim, J. P.: elastix: a toolbox for intensity-based medical image registration. IEEE Transactions on Medical Imaging 29(1), 196-205 (January 2010)
[14] Knoll, F., Murrell, T., Sriram, A., Yakubova, N., Zbontar, J., Rabbat, M., Defazio, A., Muckley, M. J., Sodickson, D. K., Zitnick, C. L., Recht, M. P.: Advancing machine learning for MR image reconstruction with an open competition: Overview of the 2019 fastMRI challenge. Magnetic Resonance in Medicine 84(6), 3054-3070 (June 2020).
   www(DOT)doi(DOT)org/10.1002/mrm.28338,
   www(DOT)doi(DOT)org/10.1002/mrm.28338
[15] Kupyn, O., Budzan, V., Mykhailych, M., Mishkin, D., Matas, J.: Deblurgan: Blind motion deblurring using conditional adversarial networks. In: 2018 IEEE/CVF Conference on Computer Vision and Pattern Recognition. pp. 8183-8192 (2018).
   www(DOT)doi(DOT)org/10.1109/CVPR.2018.00854
[16] Kupyn, O., Martyniuk, T., Wu, J., Wang, Z.: Deblurgan-v2: Deblurring (orders-of-magnitude) faster and better. In: The IEEE International Conference on Computer Vision (ICCV) (October 2019)
[17] Ledig, C., Theis, L., Huszar, F., Caballero, J., Cunningham, A., Acosta, A., Aitken, A., Tejani, A., Totz, J., Wang, Z., Shi, W.: Photo-realistic single image super-resolution using a generative adversarial network (2016).
   www(DOT)doi(DOT)org/10.48550/ARXIV.1609.04802,
   www(DOT)arxiv(DOT)org/abs/1609.04802
[18] Loshchilov, I., Hutter, F.: Sgdr: Stochastic gradient descent with warm restarts (2016)
   www(DOT)doi(DOT)org/10.48550/ARXIV.1608.03983,
   www(DOT)arxiv(DOT)org/abs/1608.03983
[19] Lustig, M., Donoho, D., Pauly, J. M.: Sparse mri: The application of compressed sensing for rapid mr imaging. Magnetic Resonance in Medicine 58(6), 1182-1195 (2007)
   www(DOT)doi(DOT)org/10.1002/mrm.21391
[20] Lustig, M., Donoho, D. L., Santos, J. M., Pauly, J. M.: Compressed sensing mri. IEEE Signal Processing Magazine 25(2), 72-82 (2008).
   www(DOT)doi(DOT)org/10.1109/MSP.2007.914728
[21] M, M.: Anisotropic neural deblurring for mri acceleration. International Journal of Computer Assisted Radiology and Surgery 17(2), 315-327 (2022).
   www(DOT)doi(DOT)org/10.1007/s11548-021-02535-6
[22] Martin, D., Fowlkes, C., Tal, D., Malik, J.: A database of human segmented natural images and its application to evaluating segmentation algorithms and measuring ecological statistics. In: Proceedings Eighth IEEE International Conference on Computer Vision. ICCV 2001. vol. 2, pp. 416-423 vol. 2 (2001).
   www(DOT)doi(DOT)org/10.1109/ICCV.2001.937655
[23] Mei, Y., Fan, Y., Zhang, Y., Yu, J., Zhou, Y., Liu, D., Fu, Y., Huang, T. S., Shi, H.: Pyramid attention networks for image restoration (2020).
   www(DOT)doi(DOT)org/10.48550/ARXIV.2004.13824,
   www(DOT)arxiv(DOT)org/abs/2004.13824
[24] Pham, C. H., Tor-D'iez, C., Meunier, H., Bednarek, N., Fablet, R., Passat, N., Rousseau, F.: Multiscale brain MRI super-resolution using deep 3D convolutional networks. Computerized Medical Imaging and Graphics 77, 101647 (2019).
   www(DOT)doi(DOT)org/10.1016/j.compmedimag.2019.101647
[25] Shamonin, D. P., Bron, E. E., Lelieveldt, B. P., Smits, M., Klein, S., Staring, M.: Fast parallel image registration on cpu and gpu for diagnostic classification of alzheimer's disease. Frontiers in Neuroinformatics 7(50), 1-15 (January 2014)
[26] Simonyan, K., Zisserman, A.: Very deep convolutional networks for large-scale image recognition (2014).
   www(DOT)doi(DOT)org/10.48550/ARXIV.1409.1556,
   www(DOT)arxiv(DOT)org/abs/1409.1556
[27] Tsiligianni, E.: Interpretable deep learning for multimodal super-resolution of medical images. In: Medical Image Computing and Computer Assisted Intervention—MICCAI 2021. pp. 421-429. Springer International Publishing (2021)
[28] Wang, Z., Bovik, A., Sheikh, H., Simoncelli, E.: Image quality assessment: from error visibility to structural similarity. IEEE Transactions on Image Processing 13(4), 600-612 (2004).
   www(DOT)doi(DOT)org/10.1109/TIP.2003.819861
[29] Xiang, L., Chen, Y., Chang, W., Zhan, Y., Lin, W., Wang, Q., Shen, D.: Deep-learning-based multi-modal fusion for fast mr reconstruction. IEEE Transactions on Biomedical Engineering 66(7), 2105-2114 (2019).
   www(DOT)doi(DOT)org/10.1109/TBME.2018.2883958
[30] Yang, G., Yu, S., Dong, H., Slabaugh, G., Dragotti, P. L., Ye, X., Liu, F., Arridge, S., Keegan, J., Guo, Y., Firmin, D.: Dagan: Deep de-aliasing generative adversarial networks for fast compressed sensing mri reconstruction. IEEE Transactions on Medical Imaging 37(6), 1310-1321 (2018).
   www(DOT)doi(DOT)org/10.1109/TMI.2017.2785879
[31] Zeyde, R.: On single image scale-up using sparse-representations. In: Curves and Surfaces. pp. 711-730. Springer Berlin Heidelberg (2012)
[32] Zhou, B., Zhou, S. K.: Dudornet: Learning a dual-domain recurrent network for fast mri reconstruction with deep t1 prior (2020).
   www(DOT)doi(DOT)org/10.48550/ARXIV.2001.03799

1. A method of magnetic resonance imaging (MRI), comprising:
acquiring a first MRI scan having a first type of MR image contrast according to a first anisotropic acquisition matrix having less elements along a first direction than along a second direction, said first direction being a phase-encoding direction for said first MRI scan, and acquiring a second MRI scan having a second type of MR image contrast according to a second anisotropic acquisition matrix having less elements along said second direction than along said first direction, said second direction being a phase-encoding direction for said second MRI scan;
feeding a machine learning procedure trained to deblur MR images with input data describing said first and said second MRI scans; and
receiving from an output of said machine learning procedure output data corresponding to a deblurred MR image.

2. The method according to claim 1, comprising, prior to said feeding, co-registering said first and said second MRI scans.

3. The method according to claim 1, wherein said first and said second MRI scans are acquired, respectively, before and after administration of a contrast agent to the object.

4. The method according to claim 1, wherein said first type of MR image contrast is different from said second type of MR image contrast, and wherein each of said first and said second type of MR image contrasts is selected from the group consisting of a T1-Weighted MR image contrast, a T2-Weighted MR image contrast, a Proton Density Weighted MR image contrast, a Fluid-Attenuated Inversion Recovery MR image contrast, a Diffusion-Weighted MR image contrast, and an Inversion Recovery MR image contrast.

5. The method according to claim 1, wherein a number of elements of said first anisotropic acquisition matrix along said second direction equals a number of elements of said second anisotropic acquisition matrix along said first direction.

6. The method according to claim 1, wherein a number of elements of said first anisotropic acquisition matrix along said first direction equals a number of elements of said second anisotropic acquisition matrix along said second direction.

7. The method according to claim 1, wherein each of said first and said second anisotropic acquisition matrix are three-dimensional and comprise elements also along a third direction, said third direction being a phase-encoding direction.

8-9. (canceled)

10. The method according to claim 1, wherein each of said first and said second anisotropic acquisition matrices are three-dimensional and comprise elements also along a third direction, wherein the method comprises acquiring a third MRI scan having a third type of MR image contrast according to a third anisotropic three-dimensional acquisition matrix having fewest elements along said third direction among said first, said second and said third three dimensional acquisition matrices, and wherein said input data also describe said third MRI scan.

11. The method according to claim 1, comprising receiving from said machine learning procedure output data corresponding to two or more deblurred MR images, at least one deblurred MR image for each of said MR scan.

12-13. (canceled)

14. A method of magnetic resonance imaging (MRI), comprising:
acquiring a first MRI scan having a first type of MR image contrast according to a first radial acquisition protocol, and acquiring a second MRI scan having a second type of MR image contrast according to a second radial acquisition protocol, wherein radial sampling lines describing said first radial acquisition protocol are interlaced with radial sampling lines describing said second radial acquisition protocol;
feeding a machine learning procedure trained to deblur MR images with input data describing said first and said second MRI scans; and
receiving from an output of said machine learning procedure output data corresponding to a deblurred MR image.

15. The method according to claim 14, wherein said interlacing forms a radial pattern having a uniform angular spacing between adjacent radial sampling lines of said pattern.

16. The method according to claim 14, wherein said first and said second MRI scans are acquired, respectively, before and after administration of a contrast agent to the object.

17. The method according to claim 14, wherein said first type of MR image contrast is different from said second type of MR image contrast, and wherein each of said first and said second type of MR image contrasts is selected from the group consisting of a T1-Weighted MR image contrast, a T2-Weighted MR image contrast, a Proton Density Weighted MR image contrast, a Fluid-Attenuated Inversion Recovery MR image contrast, a Diffusion-Weighted MR image contrast, and an Inversion Recovery MR image contrast.

18. The method according to claim 14, comprising receiving from said machine learning procedure output data corresponding to two or more deblurred MR images, at least one deblurred MR image for each of said MR scan.

19. (canceled)

20. The method according to claim 14, comprising acquiring at least one additional MRI scan having at least one respective type of MR image contrast according to a respective at least one additional radial acquisition protocol, wherein radial sampling lines describing each of said first, said second, and said at least one additional radial acquisition protocols, are all interlaced cyclically thereamongst, wherein said input data also describe said at least one additional MRI scan.

21-27. (canceled)

28. A computer software product, comprising a computer-readable medium in which program instructions are stored, which instructions, when read by an image processor, cause the data processor to receive input data, to feed a machine learning procedure trained to deblur MR images with said input data, and to receive from an output of said machine learning procedure output data corresponding to a deblurred MR image;
wherein said input data comprises data describing a first MRI scan having a first type of MR image contrast according to a first anisotropic acquisition matrix having less elements along a first direction than along a second direction, said first direction being a phase-encoding direction for said first MRI scan, and a second MRI scan having a second type of MR image contrast according to a second anisotropic acquisition matrix having less elements along said second direction than along said first direction, said second direction being a phase-encoding direction for said second MRI scan.

29. The computer software product according to claim 28, wherein a number of elements of said first anisotropic acquisition matrix along said second direction equals a number of elements of said second anisotropic acquisition matrix along said first direction.

30. The computer software product according to claim 28, wherein a number of elements of said first anisotropic acquisition matrix along said first direction equals a number of elements of said second anisotropic acquisition matrix along said second direction.

31. The computer software product according to claim 28, wherein each of said first and said second anisotropic acquisition matrix are three-dimensional and comprise elements also along a third direction, said third direction being a phase-encoding direction.

32-38. (canceled)

39. A magnetic resonance imaging (MRI) system for imaging an object, the system comprising:
an MRI scanner configured for scanning the object to provide MRI scans; and
an image processor configured for executing the method according to claim 1.
